(19) United States
(12) Patent Application Publication
(10) Pub. No.: US 2022/0370191 A1
(43) Pub. Date: Nov. 24, 2022

(54) SEMI-STABLE NEAR-FIELD ELECTROSPUN SCAFFOLDS AND METHODS OF MAKING AND USING THE SAME

(71) Applicant: The University of Memphis Research Foundation, Memphis, TN (US)

(72) Inventors: Gary Lee Bowlin, Collierville, TN (US); William Elbert King, III, Chattanooga, TN (US)

(73) Assignee: The University of Memphis Research Foundation, Memphis, TN (US)

(21) Appl. No.: 17/747,453

(22) Filed: May 18, 2022

Related U.S. Application Data
(60) Provisional application No. 63/190,135, filed on May 18, 2021.

(51) Int. Cl. A61F 2/06; D04H 1/728

(52) U.S. Cl. CPC A61F 2/06; D04H 1/728; D10B 2509/06; A61F 2210/0076; A61F 2240/001; A61F 2250/0067; A61F 2002/0086

(57) ABSTRACT

Methods of producing hybrid fibrous scaffolds are provided. The methods include dissolving a polymer, such as polydioxanone, in a solution, such as 1,1,1,3,3,3-hexafluoro-2-propanol (HFP), to form a polymer-containing solution. The method comprises electrically charging the polymer-containing solution. The method comprises writing the polymer-containing solution on a counter electrode or a ground in a grid pattern to form semi-stable fibers comprised of the polymer, the semi-stable fibers vary between bent and straight and forming the hybrid fibrous scaffold. The writing may be performed by a 3D printer. The resulting scaffolds and methods of using the same are also disclosed herein.

This application claims the benefit of U.S. provisional application No. 63/190,135, filed May 18, 2021, the contents of which is incorporated herein by reference in its entirety.

### FIELD OF DISCLOSURE

The present disclosure pertains to fibrous scaffolds. More specifically, the present disclosure is directed to semi-stable near-filed electrospun hybrid fibrous scaffolds for promoting tissue or cell regeneration and methods of making and using the same.

### BACKGROUND OF THE DISCLOSURE

Traditional electrospinning (TES) is a popular method for creating highly porous graft scaffolds to facilitate cellular ingrowth, finding many uses in vitro and in vivo. However, TES-created scaffolds (often referred to herein simply as “TES”) produce scaffolds with random architectures and have fiber diameters and pore sizes that, while tailorable, are intrinsically related to one another so that an adjustment of one affects the other. Thus, this relationship between fiber diameter and pore size in TES severely limits the range of fiber diameters to those that produce large pore sizes. Even so, these pores are still restrictively under the 60-200-micron size pore for facilitating angiogenesis. Alternatively, the relatively recent sub-technique of creating fibrous scaffolds through near-field electrospinning (NFES) takes the air gap distance of TES and shortens it to a few millimeters. This reduced air gap is then paired with precise relative motion between the charged polymer capillary and the grounded collector allowing for the direct writing of fibers. Therefore, this technique allows for electrospun fibers to be further tailorable, as individual fibers can be programmed with great precision to form 3D scaffolds that are laid down layer-by-layer. However, NFES often produces scaffolds consisting of straight-line fibers that lack the random fibrous qualities, which when beneficially present, promote cellular growth within the scaffold.

Meanwhile, cardiovascular disease (CVD) is a growing condition caused by narrowing blood vessels in a process termed atherosclerosis. Current surgical interventions when less invasive options are no longer viable include bypass surgery with either autografts or “off-the-shelf” manufactured grafts. Autologous sources of vascular replacements are often limited because of disease or the necessity for multiple replacements per patient. Current manufactured, off-the-shelf products such as DACRON® polyethylene terephthalate (referred to herein simply as “DACRON®”) or GORE-TEX® expanded polytetrafluoroethylene (ePTFE) (referred to herein simply as “GORE-TEX®”) work adequately in adults for graft inner diameters larger than 6 mm, but smaller diameters experience high failure rates. In the short term, these existing grafts fail due to thrombosis and issues stemming from mechanical property mismatch. Therefore, there is an unmet need for a scaffold that can have a small diameter, be off-the-shelf, and be bioresorbable to serve as a graft. To address these shortcomings, the ideal solution would be an off-the-shelf, bioresorbable graft that would allow for blood to flow without thrombus formation to profuse downstream tissues while serving as a template to guide in situ regeneration of a functional artery.

### BRIEF SUMMARY

The problems described above, as well as others, are addressed by the following embodiments, although it is to be understood that not every embodiment of this disclosure will address each of the problems described above. Further advantages, features, and details of the embodiments can be gathered from the claims, the description of preferred embodiments below, as well as the drawings.

The present disclosure relates to scaffolds, methods for producing scaffolds, and uses of scaffolds. In particular, disclosed herein are methods for making a hybrid fibrous scaffold comprising the steps of: dissolving a polymer in a solution to create a polymer-containing solution and writing the polymer-containing solution on a counter electrode to form semi-stable fibers comprised of the polymer.

The method for producing a hybrid fibrous scaffold comprises dissolving a polymer in a solution. The hybrid fibrous scaffold may mimic an extracellular matrix of a subject. In some embodiments, the polymer is polydioxanone. Any suitable solution may be used for dissolving the polymer. In some embodiments, the solution is a solvent, or more specifically, 1,1,1,3,3,3-hexafluoro-2-propanol (HFP). The polymer may be dissolved in the solution to a concentration, by weight volume in the solution, to any suitable concentration, such as a concentration of from 25 mg/mL to 450 mg/mL, from 50 mg/mL to 225 mg/mL, or 112 mg/mL.

The method of producing a hybrid fibrous scaffold can comprise electrically charging the polymer-containing solution.

In embodiments, the method of producing a hybrid fibrous scaffold comprises writing the polymer-containing solution on a counter electrode or a ground in a grid pattern to form semi-stable fibers comprised of the polymer, the semi-stable fibers comprising bent fibers and straight fibers and forming the fibrous scaffold. The semi-stable fibers can comprise a plurality of bent fibers and a plurality of straight fibers. The writing may be performed in layers such that the hybrid fibrous scaffold has from 10 layers to 10,000 layers, from 50 layers to 600 layers, or from 100 layers to 300 layers. The number of layers in the scaffold may be equal to the number of layers written. The writing may be performed in layers to form a stable layer and an unstable layer. In some embodiments, the stable layer comprises straight fibers and the unstable layer comprises bent fibers. The stable layer can be adjacent to the unstable layer. The stable layer and the unstable layer may be written sequentially at least two times or at least twenty times. In embodiments, switching between a stable layer and an unstable layer is a random, stochastic process. The writing may be performed by an additive manufacturing system, such as a 3D printer. Fiber placement may be programmed in the additive manufacturing system such that the additive manufacturing system deposits solution based on the programmed fiber placement and fiber stability. In some embodiments, the step of electrically charging the polymer-containing solution comprises exposing the polymer-containing solution to an applied voltage and the step of writing the polymer-containing solution comprises setting an air gap distance. The method can further comprise increasing the number of bent fibers by increasing the applied voltage, the air gap distance, or a combination thereof.

The polymer-containing solution may be written on a counter electrode or ground in a predetermined writing path comprising one or more of: a grid size, a scaffold size, a layer count, an air gap, an electric field strength, and a geometry. The counter electrodes or grounds may have any suitable surface geometry. That is, the surface on which the solution is deposited may be flat, concave, convex, irregular, or have any other geometry. The grid size may be up to from 50 μm×50 μm to 2,000 μm to 2,000 μm, from 100 μm×100 μm to 1,000 μm to 1,000 μm, or from 200 μm×200 μm to 500 μm to 500 μm. The scaffold size may be from 20 mm×5 mm to 400 mm to 100 mm, from 40 mm×10 mm to 200 mm to 25 mm, or from 84 mm×22 mm to 60 mm×60 mm, or up to 10,000 mm×10,000 mm.

In embodiments, the degree of fiber stability depends, at least in part, on the electric field strength during writing of the semi-stable fibers. Fiber stability can be inversely proportional to electric field strength. Increasing the applied voltage during writing of the semi-stable fibers causes an increase in the electric field strength. In embodiments, semi-stable fibers are made more unstable by increasing applied voltage thereby increasing the electric field strength and increasing the air gap distance to accentuate bending instabilities. The degree of fiber stability can be inversely proportional to air gap distance, the applied voltage, or a combination thereof. The rate of bent fiber formation can be directly proportional to the air gap distance, the applied voltage, or a combination thereof. In some embodiments, increasing the air gap distance at a given applied voltage reduces fiber stability and increases the rate of bent fiber formation. Increasing the applied voltage at a given air gap distance can reduce fiber stability and increase the rate of bent fiber formation. The air gap may be from 1 mm to 10 mm or 2 mm to 5 mm. In some embodiments, the air gap is about 3 mm. As used consistent with its meaning in the art, the “air gap” is the distance between the polymer source (e.g., a print head) and the collector (also referred to as the counter electrode). The applied voltage may be from 0.1 kV to 10.0 kV. In embodiments, the applied voltage is between 0.5 kV and 5.0 kV. The applied voltage may be between 0.9 kV and 3 kV. In embodiments, the applied voltage comprises 1.0 kV, 1.1 kV, 1.2 kV, 1.3 kV, 1.4 kV, 1.5 kV, 1.6 kV, 1.7 kV, 1.8 kV, 1.9 kV, or 2.0, 2.1 kV, 2.2 kV, 2.3 kV, 2.4 kV, 2.5 kV, 2.6 kV, 2.7 kV, 2.8 kV, 2.9 kV, or 3.0 kV. In some embodiments, the electric field strength is from 0.1 kV/mm to 2.0 kV/mm. The electric field strength can be from 0.5 kV/mm to 1.8 kV/mm. In embodiments, the electric field strength is from 0.7 kV/mm to 1.7 kV/mm. The electric field strength can be 0.1 kV/mm, 0.2 kV/mm, 0.3 kV/mm, 0.4 kV/mm, 0.5 kV/mm, 0.6 kV/mm, 0.7 kV/mm, 0.8 kV/mm, 0.9 kV/mm, 1.0 kV/mm, 1.1 kV/mm, 1.2 kV/mm, 1.3 kV/mm, 1.4 kV/mm, 1.5 kV/mm, 1.6 kV/mm, 1.7 kV/mm, 1.8 kV/mm, 1.9 kV/mm, or 2.0 kV/mm.

The layer count may comprise the number of layers in which the scaffold was written (e.g., from 10 layers to 10,000 layers, from 50 layers to 600 layers, or from 100 layers to 300 layers). The geometry may comprise a stacking grid geometry. The layers may each have a predetermined writing path that is the same as to the other layer(s) or different as to other of the layer(s).

One or more of the semi-stable fibers may have an average diameter of from 0.1 μm to 10 μm, from 0.5 μm to 4 μm, or from 1 μm to 2 μm. The hybrid fibrous scaffold may have a thickness of from 0.01 mm to 1 mm, 0.05 mm to 0.5 mm, or from 0.09 mm to 0.12 mm. The hybrid fibrous scaffold may have an average surface pore size of from 1 μm to 200 μm, from 15 μm to 200 μm, or from 1 μm to 56.8 μm. The hybrid fibrous scaffold may have a 90^{th} percentile scaffold pore size of greater than 25 μm, greater than 37 μm, greater than 40 μm, greater than 50 μm, less than 200 μm, less than 100 μm, less than 75 μm, or any range or subvalue between any of the foregoing. The present disclosure is also directed towards hybrid fibrous scaffolds produced by the methods addressed herein. That is, disclosed in various embodiments herein are fibrous scaffolds comprising polymer semi-stable fibers that vary between bent and straight. In certain embodiments, a plurality of straight fibers are aligned to form a stacking grid geometry with a programmed grid spacing and a plurality of bent fibers extending across at least a portion of the programmed grid spacing. The hybrid fibrous scaffolds may release (including extendedly release) one or more therapeutic agents. The hybrid fibrous scaffolds may have highly aligned grid fibers. The hybrid fibrous scaffolds may comprise a vascular graft scaffold for promoting or facilitating transmural capillary cellular growth. The hybrid fibrous scaffold may have a permeability to 9.9 μm microspheres of from 150 microspheres/mm² to 3000 microspheres/mm² or from 243 microspheres/mm² to 1603 microspheres/mm². The hybrid fibrous scaffold may have a permeability to 97 μm microspheres of from 1 microspheres/mm² to 5 microspheres/mm² or from 1 microspheres/mm² to 3 microspheres/mm². In some embodiments, the hybrid fibrous scaffold comprises a low density of random or bent fibers when the hybrid fibrous scaffold is permeable to 97 μm microspheres. A hybrid fibrous scaffold with a low density of random or bent fibers can have a permeability to 97 μm microspheres of from 1 microspheres/mm² to 5 microspheres/mm² or from 1 microspheres/mm² to 3 microspheres/mm². In certain embodiments, a hybrid fibrous scaffold with a low density of random or bent fibers can have a permeability to 9.9 μm microspheres of from 150 microspheres/mm² to 3000 microspheres/mm² or from 243 microspheres/mm² to 1603 microspheres/mm². In some embodiments, a low density of random or bent fibers can occur when less than 50% of the fibers in the hybrid fibrous scaffold are random or bent. In other embodiments, a hybrid fibrous scaffold with a low density of random or bent fibers can be a hybrid fibrous scaffold wherein less than 30% of the fibers are bent or random. In still other embodiments, a hybrid fibrous scaffold comprises a low density of random or bent fibers when less than 20%, less than 10%, less than 5%, less than 4%, less than 3%, less than 2%, or less than 1% of the semi-stable fibers are random or bent.

The hybrid fibrous scaffolds of the present disclosure find use in therapeutic methods or uses, such as implant material scaffolds for cardiovascular tissue regeneration, musculoskeletal tissue regeneration, cancer therapies, immunotherapies such as using a scaffold to create an artificial thymus, or preventing or treating disease or injury in a subject (e.g., an animal or a human). The scaffolds may be administered (e.g., directly or indirectly) to a target tissue or organ (such as one that is damaged or diseased) to establish functional connections to regenerate or treat the target tissue or organ. The scaffolds may be applied to contact (e.g., cover, surround, or fill) a bone or tissue defect, a wound, or a surgical site.

In one embodiment, the scaffolds enhance tissue regeneration, such as soft tissue (e.g., cardiovascular tissue or cells). Methods for repairing damaged tissue or organs may be carried out either in vitro, in vivo, or ex vivo.

In another embodiment, the disclosure provides a method of promoting endothelialization in a subject, the method comprising implanting the scaffolds as a vascular graft in the subject.

In another embodiment, the disclosure provides a vascular graft comprising the scaffolds of the disclosure.

The foregoing methods are not exclusive. One or more methods may be employed concurrently using one or more embodiments of the scaffolds.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present concepts in a simplified form as a prelude to the more detailed description that is presented later.

### BRIEF DESCRIPTION OF THE DRAWINGS

FIGS. 1A-1F. FIG. 1A shows a scanning electron micrograph of TES_{1-2 μm} scaffold. FIG. 1B shows a scanning electron micrograph of a NFES 200² scaffold. FIG. 1C shows a scanning electron micrograph of a NFES 300² scaffold. FIG. 1D shows a scanning electron micrograph of a NFES 400² scaffold. FIG. 1E shows a scanning electron micrograph of a NFES 500² scaffold. FIG. 1F illustrates surface pore measurements of the scaffolds of FIGS. 1A-1E (* indicates p<0.05, Scale bar=200 μm).

FIGS. 2A-2B. FIG. 2A illustrates microsphere filtration of the scaffolds shown in FIGS. 1A-1E for 9.9 μm spheres (* indicates p<0.05). FIG. 2B illustrates microsphere filtration of the scaffolds shown in FIGS. 1A-1E for 97 μm spheres (* indicates p<0.05).

FIGS. 3A-3B. FIG. 3A illustrates ultimate tensile strength of the scaffolds shown in FIGS. 1A-1E in the principal axis (* indicates p<0.05, NS indicates no significances). FIG. 3B illustrates ultimate tensile strength of the scaffolds shown in FIGS. 1A-1E in the 45° axis (* indicates p<0.05, NS indicates no significances).

FIGS. 4A-4B. FIG. 4A illustrates percent elongation at failure of the scaffolds shown in FIGS. 1A-1E in the principal axis (* indicates p<0.05). FIG. 4B illustrates percent elongation at failure of the scaffolds shown in FIGS. 1A-1E in the 45° axis (* indicates p<0.05).

FIGS. 5A-5B. FIG. 5A illustrates yield stress of the scaffolds shown in FIGS. 1A-1E for the principal axis (* indicates p<0.05, NS indicates no significances). FIG. 5B illustrates yield stress of the scaffolds shown in FIGS. 1A-1E for the 45° axis (* indicates p<0.05, NS indicates no significances).

FIGS. 6A-6B. FIG. 6A illustrates percent elongation at yield for the scaffolds shown in FIGS. 1A-1E for the principal axis (* indicates p<0.05). FIG. 6B illustrates percent elongation at yield for the scaffolds shown in FIGS. 1A-1E for the 45° axis (* indicates p<0.05).

FIGS. 7A-7B. FIG. 7A illustrates Young's modulus for the scaffolds shown in FIGS. 1A-1E for the principal axis (* indicates p<0.05). FIG. 7B illustrates Young's modulus for the scaffolds shown for FIGS. 1A-1E in the 45° axis (* indicates p<0.05).

FIGS. 8A-8B. FIG. 8A illustrates the percent area covered by NETs at 3 hours (* indicates p<0.05). FIG. 8B illustrates the percent area covered by NETs at 6 hours (* indicates p <0.05).

FIGS. 9A-9L. FIGS. 9A-9F show representative fluorescence microscopy images of NETs for the scaffolds shown in FIGS. 1A-1E at 3 hours. FIGS. 9G-9L show representative fluorescence microscopy images of NETs for the scaffolds shown in FIGS. 1A-1E at 6 hours. Scale bar=100 μm, Blue=DAPI, Green=Actin Green, Red=Sytox Orange.

FIGS. 10A-10B. FIG. 10A shows a scanning electron micrograph of a scaffold of the present disclosure having small grids (scale bar=200 μm). FIG. 10B shows a scanning electron micrograph of a scaffold of the present disclosure having large grids (scale bar=200 μm).

FIGS. 11A-11F. FIGS. 11A-11F show qualitative digital microscopy images of a GORE-TEX® scaffold (FIG. 11A), a TES scaffold (FIG. 11B), a NFES 200² scaffold (FIG. 11C), a NFES 500² scaffold (FIG. 11D), a 45°/45° vascular graft (FIG. 11E), and a 20°/70° vascular graft (FIG. 11F).

FIGS. 12A-12B. FIGS. 12A-12F shows a scanning electron micrograph of a GORE-TEX® scaffold (FIG. 12A), a TES scaffold (FIG. 12B), a NFES 200² scaffold (FIG. 12C), a NFES 500² scaffold (FIG. 12D), a 45°/45° vascular graft (FIG. 12E), and a 20°/70° vascular graft (FIG. 12F). Scale bar=200 μm.

FIGS. 13A-13B. FIGS. 13A-13B illustrate fluorescent microsphere permeability of vascular grafts for 9.9 μm microspheres (FIG. 13A) and 97 μm microspheres (FIG. 13B). * indicates p<0.05.

FIGS. 14A-14B. FIGS. 14A-14B illustrate ultimate tensile strength of vascular grafts in the circumferential axis (FIG. 14A) and the longitudinal axis (FIG. 14B). * indicates p<0.05, black dotted line indicates literature value for internal mammary artery (IMA).

FIGS. 15A-15B. FIGS. 15A-15B illustrate percent elongation at failure of vascular grafts in the circumferential axis (FIG. 15A) and the longitudinal axis (FIG. 15B). * indicates p<0.05, black dotted line indicates literature value for IMA.

FIG. 16. FIG. 16 illustrates single wall 90° cut suture retention of vascular grafts (* indicates p<0.05, black dotted lines indicate the range of literature values for IMA).

FIG. 17. FIG. 17 illustrates burst pressure of vascular grafts (* indicates p<0.05, black dotted line indicates literature value for IMA, red solid line indicates apparatus measurement maximum).

FIGS. 18A-18B. FIGS. 18A-18B illustrate Young's modulus of vascular grafts in the circumferential axis (FIG. 18A) and representative stress-strain curves of vascular grafts (FIG. 18B). NS indicates p>0.05 with all other comparisons being p<0.05, black dotted line indicates literature value for IMA.

FIGS. 19A-19D. FIGS. 19A-19B illustrate platelet adhesion on the lumen of vascular grafts as actin cytoskeleton surface average at 15 minutes (FIG. 19A) and 30 minutes (FIG. 19B). FIGS. 19C-19D illustrate P-selectin surface expression coverage at 15 minutes (FIG. 19C) and 30 minutes (FIG. 19D). * indicates p<0.05, NS indicates p>0.05 with all other comparisons being p<0.05.

FIGS. 20A-20P. FIGS. 20A-20H show representative fluorescence microscopy images of adhered platelets on GORE-TEX® (FIG. 20A), TES (FIG. 20B), NFES 200² (FIG. 20C), NFES 500² (FIG. 20D), NFES 45°/45° (FIG. 20E), NFES 20°/70° (FIG. 20F), TES+PMA (FIG. 20G), and TES+Vehicle Control (FIG. 20H) at 15 minutes. FIGS. 201-20P show representative fluorescence microscopy images of adhered platelets on GORE-TEX® (FIG. 20I), TES (FIG. 20J), NFES 200² (FIG. 20K), NFES 500² (FIG. 20L), NFES 45°/45° (FIG. 20M), NFES 20°/70° (FIG. 20N), TES+PMA (FIG. 20O), and TES+Vehicle Control (FIG. 20P) at 30 minutes. Scale bar=100 μm, Blue=DAPI, Green=Actin Green, Red=P-Selectin.

### DETAILED DESCRIPTION

The present invention features scaffolds and methods of making and using the same. Reference now will be made in detail to the embodiments of the present disclosure. It is understood that the invention is not limited to the particular methodology, protocols, and reagents, etc., described herein, as these can be varied by one of ordinary skill in the art. It is also understood that the terminology used herein is used for the purpose of describing particular illustrative embodiments only and is not intended to limit the scope of the invention. It should be noted that the features illustrated in the drawings are not necessarily drawn to scale, and without departing from the scope of the disclosure, features of one embodiment may be employed with other embodiments as those of ordinary skilled in the art would recognize, even if not explicitly stated herein. For instance, features illustrated or described as part of one embodiment, can be used with another embodiment to yield a further embodiment. Descriptions of well-known components and processing techniques may be omitted so as to not unnecessarily obscure the embodiments of the disclosure.

Thus, it is intended that the present disclosure covers such modifications and variations as come within the scope of the appended claims and their equivalents. Other objects, features, and aspects of the present disclosure are disclosed in or are apparent from the following detailed description. It is to be understood by one of ordinary skill in the art that the present disclosure is a description of exemplary embodiments only and is not intended as limiting the broader aspects of the present disclosure.

### I. Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense, unless expressly so defined herein. Well-known functions or constructions may not be described in detail for brevity or clarity. As used herein, the following terms have the meanings ascribed to them below, unless specified otherwise.

Unless specifically stated or obvious from context, the term “or” is understood to be inclusive. The singular forms “a,” “an,” and “the” include the plural reference unless the context clearly dictates otherwise. Thus, for example, a reference to “a fiber” is a reference to one or more fibers and equivalents thereof known to those of ordinary skill in the art.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. The term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

As used herein, terms such as “administering” or “administration” include acts such as prescribing, dispensing, giving, or taking a substance such that what is prescribed, dispensed, given, or taken actually contacts the patient's body externally or internally (or both). In embodiments of this disclosure, terms such as “administering” or “administration” include self-administering, self-administration, and the like, of a substance. Indeed, it is specifically contemplated that instructions or a prescription by a medical professional to a subject or patient to take or otherwise self-administer a substance is an act of administration.

As used herein, an “agent” means any molecule chemical compound, antibody, nucleic acid molecule, or polypeptide, or fragments thereof, including any compounds commonly known as a “drug.”

As used herein, “ameliorate” means decrease, suppress, attenuate, diminish, arrest, or stabilize the development or progression of a disease.

In this disclosure, “comprises,” “comprising,” “containing,” “including,” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “disease” means any condition or disorder that damages or interferes with the normal function of a cell, tissue, or organ, including bone.

As used herein, “enhancing tissue regeneration” or “promoting tissue regeneration” or the like means increasing the extent of growth or healing relative to a control condition.

As used herein, the terms “prevention,” “prevent,” “preventing,” “suppression,” “suppress,” and “suppressing” refer to a course of action (such as administering a scaffold) initiated prior to the onset, amplification, or exacerbation of a clinical manifestation of a disease state or condition so as to reduce its likelihood or severity. Such reduction in likelihood or severity need not be absolute to be useful.

As used herein, “subject” means a mammal, including, but not limited to, a human or non-human mammal, such as a bovine, equine, canine, ovine, or feline.

In some embodiments, “straight fiber” refers to a subset of semi-stable fibers that are substantially straight. In further embodiments, “straight fibers” can refer to highly ordered stacked fibers and highly aligned grid fibers. In certain embodiments, straight fibers comprise a morphology or configuration similar to that of fibers generated through NFES.

As used herein, a “stable layer” can refer to a layer of the hybrid fibrous scaffold that comprises one or more straight fibers.

The terms “bent fiber” and “random fiber” may be used interchangeably herein. In embodiments, “bent fiber” and “random fiber” refer to fibers that are chaotically bent or bent according to a stochastic process. In certain embodiments, bent fibers and random fibers comprise a morphology or a configuration that is similar to that of fibers generated through TES. Bent fibers and random fibers can refer to the fibers that create the random fiber infill within the highly aligned grid structures disclosed herein.

As used herein, an “unstable layer” can refer to a layer of the hybrid fibrous scaffold that comprises one or more bent fibers.

“Fiber stability,” as used herein, can refer to the degree of bending instabilities that occurs when semi-stable fibers are written in accordance with various embodiments disclosed herein. By way of example, conditions that increase fiber stability can refer to parameters that reduce the number of bending instabilities. For example, an increase in fiber stability may be observed by decreasing the applied voltage when the semi-stable fiber is being written. Similarly, an increased fiber stability may occur when the semi-stable fibers are written with a smaller air gap distance. That is to say, in certain embodiments, fiber stability can be inversely proportional to applied voltage, air gap distance, electric field strength, or a combination thereof. In embodiments, any one or more of the following can affect fiber stability: air gap distance, applied voltage, print head translation velocity, and concentration of polymer within the polymer-containing solution. For instance, increasing the print head velocity can increase fiber stability, whereas reducing the print head speed can provide additional time for fiber bending to occur, thereby reducing fiber stability. Additionally, increasing polymer concentration can have a minor but directly proportional effect on fiber stability. In such embodiments, as the polymer concentration increases, more polymer chain entanglements occur resulting in larger fibers, and consequently, fibers that are less easily bent and more stable.

As used herein, “grid pattern,” grid scaffold,” “grid structure,” and the like can refer to a plurality of fibers that intersect one another in an ordered manner, such that a consistently shaped grid spacing exists between the intersections of the plurality of fibers. In embodiments, the grid spacing can comprise any known geometric shape. By way of example, the shape of the grid spacing can be a rhombus, a parallelogram, a trapezoid, or a combination thereof. The shape of the grid spacing can be substantially circular. In embodiments, the shape of the grid spacing is a rectangle, a square, a triangle, a pentagon, a hexagon, a heptagon, an octagon, a nonagon, a decagon, or an oval. The plurality of fibers forming the grid pattern can comprise at least two intersecting fibers. In embodiments, the plurality of fibers forming the grid pattern can comprise at least 10 fibers, at least 50 fibers, at least 100 fibers, at least 500 fibers, at least 1,000 fibers, at least 5,000 fibers, at least 10,000 fibers, at least 100,000 fibers, at least 500,000 fibers, at least 1 million fibers, at least 50 million fibers, at least 500 million fibers, at least 1 billion fibers, at least 50 billion fibers, or at least 100 billion fibers. In certain embodiments, the plurality of fibers forming the gid pattern comprises over 100 billion fibers.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The recitation of an embodiment for a variable or aspect herein includes that embodiment as any single embodiment or in combination with any other embodiments or portions thereof.

Any scaffolds or methods involving the same provided herein can be combined with one or more of any of the other scaffolds or methods involving the same provided herein.

It is to be understood that any given element of the disclosed embodiments of the invention may be embodied in a single structure, a single step, a single substance, or the like. Similarly, a given element of the disclosed embodiment may be embodied in multiple structures, steps, substances, or the like.

### II. Methods of Producing Hybrid Fibrous Scaffolds

The present disclosure relates to scaffolds, methods for producing scaffolds, and uses of scaffolds. In certain embodiments, the present disclosure provides for programmable fibrous scaffolds where fiber diameter, pore size, and mechanical properties can be independently tailored. In particular, disclosed herein are methods for making a hybrid fibrous scaffold comprising the steps of: dissolving a polymer in a solution and writing the polymer-containing solution on a counter electrode in a grid pattern to form semi-stable fibers comprised of the polymer.

The method for producing a fibrous scaffold comprises dissolving a polymer in a solution. The hybrid fibrous scaffold may mimic an extracellular matrix of a subject. The hybrid fibrous scaffold may be implantable and/or biocompatible. The hybrid fibrous scaffold may be in a solid form. Any suitable polymer may be used. The polymer may be a synthetic polymer or a natural polymer. Examples of synthetic polymers that may be utilized in the present disclosure include, but are not limited to: poly(urethane), a poly(siloxane) or a silicone, a poly(ethylene), poly(vinyl pyrrolidone), poly(-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), polydioxanone (PDO), a nylon, a polyamide, a polyanhydride, poly(ethylene-co-vinyl alcohol) (EVOH), polycaprolactone, poly(vinyl acetate) (PVA), polyvinylhydroxide, poly(ethylene oxide) (PEO), poly(2ethyl2oxazolene) (PEOZ), poly(styrene-co-acrylonitrile) (SAN), acrylonitrile butadiene styrene (ABS), polyorthoesters (PEA), a copolymer of PLA, PGA, PLGA, PDO, or PEO, or combinations thereof. In some embodiments, the polymer is polydioxanone. Non-limiting examples of natural polymers that may be utilized in the present disclosure are chitosan, collagen, elastin, alginate, cellulose, hyaluronic acid, gelatin, or combinations thereof. More than one polymer or more than one combination of polymers may be used. In certain embodiments, the polymer comprises a synthetic polymer and a natural polymer. In some embodiments, the scaffold is free of natural polymers. In other embodiments, the scaffold is free of synthetic polymers.

Any suitable solution may be used for dissolving the polymer. In some embodiments, the solution is a solvent, or more specifically, an organic solvent. Exemplary organic solvents that may be utilized in the present disclosure are 1,1,1,3,3,3-hexafluoro-2-propanol (HFP), ethanol, acetone, dimethylformamide (DMF), dimethylsulfoxide (DMSO), chloroform, 2,2,6,6-tetramethyl-heptane, or dichloromethane (DCM), or combinations thereof. The solvent can comprise any solvent known by one of skill in the art to be capable of dissolving a polymer. The polymer may be dissolved in the solution to a concentration, by weight volume of the solution, to any suitable concentration, such as a concentration of from 25 mg/mL to 450 mg/mL, from 50 mg/mL to 225 mg/mL, or 112 mg/mL. A non-limiting, exemplary method of dissolving the polymer in the solution is described in Working Example 1. The phrase “polymer-containing solution,” as used herein, can refer to the solution in which the one or more polymers have been dissolved.

In some embodiments, the method of producing a hybrid fibrous scaffold comprises electrically charging the polymer-containing solution. The method of producing a hybrid fibrous scaffold can comprise writing the polymer-containing solution on a counter electrode or a ground in a grid pattern to form semi-stable fibers comprised of the polymer, the semi-stable fibers varying between bent and straight and forming the hybrid fibrous scaffold. The counter electrode or ground may have any suitable surface geometry. The writing may be performed via any suitable additive manufacturing process, such as three-dimensional (3D) printing processes, performed by an additive manufacturing system (such as a 3D printer). The additive manufacturing process may include processes such as a polymer jet deposition process, an inkjet printing process, a fused deposition molding process, a binder jetting process, a powder bed fusing process, a selective laser sintering process, a stereolithography process, a photo-polymerization curing digital light process, a sheet lamination process, a directed energy deposition process, or other similar 3D deposition processes.

The additive manufacturing system printer may include one or more printer heads having one or more nozzles or needles for dispensing (i.e., writing) the solution, such as directly onto a counter electrode or ground. The polymer-containing solution may be charged before, or as it is written, such as with an applied voltage of 1.6 kV. The print head may have a print head translation velocity of 80 mm/s to produce fibers with a target diameter range of 1-2 μm. The counter electrode or ground may comprise an electrically grounded aluminum plate. After the print head extrudes the polymer-containing solution, the print head may be translated, such as at 10 mm/s for 60 seconds, to allow for fiber deposition to stabilize. In some embodiments, the velocity and time of the print head may be varied to accordingly vary the amount of stabilization in a printed layer. Specifically, the print head speed may be varied (such as increased or decreased) and time (increased or decreased) such that the polymer-containing solution in the printed layer may be resultingly unstable or stable. In this way, the layers may be varied as to stability, such as alternating. In some embodiments, the scaffold may be written in layers, and the layers may comprise alternating stable, semi-stable, and unstable layers. Advantageously, this stability control on a layer-by-layer basis allows for the physical properties of the scaffold to be highly tailored, as it allows for fiber geometry to be adjusted on a layer-basis. The stable layer and the unstable layer may be written sequentially at least twice, at least four times, at least ten times, at least twenty times, at least fifty times, at least one-hundred times, at least two-hundred-and-fifty times, or at least five-hundred times. The print heads may have a very fine resolution, such as a deposited solution droplet width of less than 10 μm, less than 8 μm, less than 5 μm, less than 4 μm, less than 3 μm, less than 2 μm, less than 1 μm, less than 0.5 μm, less than 0.1 μm, or less than 0.01 μm.

The solution may be deposited at selected or predetermined locations to form the fibers having desired characteristics. These selected locations may collectively form a target print pattern that can be stored as an engineering file, such as a CAD file, that is read by an electronic controller on the additive manufacturing system that controls delivery (i.e., the writing) from the one or more nozzles. Thus, fiber placement may be programmed in the engineering file in one or more predetermined locations to be read by the additive manufacturing system and cause the additive manufacturing system to deposit solution to form a fiber at those predetermined locations.

The controller may be used to facilitate control and automation of components of the additive manufacturing system, including movement of the print head and dispensing of the solution. The controller may be, for example, a computer, a programmable logic controller, an embedded controller, or combinations thereof. The controller may include a central processing unit (CPU), memory, and support circuits for input and output. The CPU may be one of any form of computer processor used in an industrial setting for controlling various system functions, substrate movement, chamber processing, and controlling support hardware (e.g., sensors, motors, heaters, etc.), and monitors the processes performed in the system. The memory is connected to the CPU and may be one or more of an off-the-shelf non-volatile memory such as Random Access Memory (RAM), flash memory, Read Only Memory (ROM), floppy disk, hard disk, or any other form of digital storage, local or remote. Software instructions and data may be encoded and stored within memory for instructing the CPU. The support circuits are also coupled to the CPU for supporting the processor in a conventional manner. The support circuits may include cache, power supplies, clock circuits, input/output circuits, subsystems, and the like. A program (or computer instructions) readable by the controller determines which tasks may be performed by components in the additive manufacturing system. The program may be software readable by the controller that includes code to perform tasks to perform and control the selective delivery, timing, and the positioning of the solution written by the additive manufacturing system. Machine-readable instructions, or parameters, may be inputted by a user (i.e., programmed) to be read, and performed, by the additive manufacturing system. The programmed instructions may include, for example, a predetermined writing path for writing the solution on the counter electrode or ground.

The predetermined writing path comprises one or more of: a grid size, a scaffold size, a layer count, an air gap, and a geometry. The grid size may be from 50×50 μm to 10,000 to 10,000 μm, from 2,000 μm×2,000 μm to 5,000 μm to 5,000 μm, from 100 μm×100 μm to 1,000 to 1,000 μm, or from 200 μm×200 μm to 500 μm to 500 μm. The scaffold size may be from 20 mm×5 mm to 400 mm to 100 mm, from 40 mm×10 mm to 200 mm to 25 mm, or from 84 mm×22 mm to 60×60 mm. The air gap may be from 1 mm to 10 mm or 2 mm to 5 mm. The air gap can be 3 mm. The layer count may comprise the number of layers in which the scaffold was written (e.g., from 10 to 10,000 layers, from 50 to 600 layers, or from 100 to 300 layers). The geometry may comprise a stacking grid geometry. The layers may each have a predetermined writing path that is the same as to the other layer(s) or different as to other of the layer(s). The additive manufacturing system may be highly precise in depositing solution based on programming. For example, the additive manufacturing system may be programmed to deposit solution in one or more programmed locations, such as over a programmed grid pattern, and the additive manufacturing system may deposit the solution in an actual location that is a distance of +/−0.01, 0.1, 0.5, 1, 2, 3, 5 or 10 μm on the ground or plate as compared to the intended location (i.e., a high degree of precision).

The grid pattern may be programmed into the additive manufacturing printer (i.e., 3D printer) to define a grid pattern for the 3D printer to extrude the polymer-containing solution. Any suitable grid pattern may be programmed. By way of example, a grid pattern may be programmed with X- and Y-grid spacing of 200×200 μm (NFES 200²), 300×300 μm (NFES 300²), 400×400 μm (NFES 400²), or 500×500 μm (NFES 500²). The one or more layers may each be printed in the same grid pattern (i.e., X- and Y-grid spacing such that the layers appear in a “stacking” grid pattern) or in offset grid patterns from one another.

As shown in Working Example 1, advantageously, when the print head was programmed to translate in a stacking grid pattern, the resulting scaffold structure was highly aligned grid fibers that were intercalated with low density, random fibers. Indeed, as the semi-stable switching process can be considered random, increasing the grid size results in both a lower density of fibers in the center of each grid cell and intercalated into the grid structure as seen on SEM imaging as well as a lower density of “rebar-like” stacked fibers. Consequently, the degree of inefficiently packed fibers intercalated in the grid structure can increase the apparent scaffold thickness for a constant fiber diameter as seen by the increasing number of layers required to achieve the same scaffold thickness for increasing grid sizes in Table 1. Therefore, the presently disclosed technique decouples the association between fiber diameter and pore size, allowing for previously unachievable tissue engineering scaffold tailoring.

The writing may be performed in layers such that the hybrid fibrous scaffold has from 10 to 10,000 layers, from 50 to 600 layers, or from 100 to 300 layers. The number of layers in the scaffold may be equal to the number of layers written. The writing may be performed by a 3D printer, such as a 3D printing head having a polymer flow rate of 25 μL/hr through a 23-gauge, 2-inch blunt needle.

The semi-stable fibers may have an average diameter of from 0.1 μm to 10 μm, from 0.5 μm to 4 μm, or from 1 μm to 2 μm. The hybrid fibrous scaffold may have a thickness of from 0.01 mm to 1 mm, 0.05 mm to 0.5 mm, or from 0.09 mm to 0.12 mm. The hybrid fibrous scaffold may have an average surface pore size of from 1 μm to 200 μm, from 15 μm to 200 μm, or from 15 μm to 56.8 μm. The hybrid fibrous scaffold may have a 90^{th} percentile scaffold pore size of greater than 25 μm, greater than 37 μm, greater than 40 μm, greater than 50 μm, or less than 200 μm, less than 100 μm, or less than 75 μm, or any range or subvalue between any of the foregoing.

Disclosed herein are hybrid fibrous scaffolds comprising polymer semi-stable fibers that vary between bent and straight. The hybrid fibrous scaffolds may release (including extendedly release) one or more therapeutic agents. The hybrid fibrous scaffolds may have highly aligned grid fibers. The hybrid fibrous scaffolds may be a vascular graft scaffold for promoting or facilitating transmural capillary cellular growth. The hybrid fibrous scaffold may have a permeability to 9.9 μm microspheres of from 150 microspheres/mm² to 3000 microspheres/mm² or from 243 microspheres/mm² to 1603 microspheres/mm². The hybrid fibrous scaffold may have a permeability to 97 μm microspheres of from 1 microspheres/mm² to 5 microspheres/mm² or from 1 microspheres/mm² to 3 microspheres/mm².

The methods may include removing the solution from the written polymer-containing solution, such as through heat drying, storing at a pressure of less than atmospheric pressure, desiccation, or combinations thereof. When the solution is a solvent, the removing may be removing the residual solvent present in the written polymer-containing solution.

The methods disclosed herein may include sterilizing the hybrid fibrous scaffolds, such as via UV sterilization, ETO sterilization, or any other suitable sterilization.

The foregoing methods are not exclusive. One or more methods may be employed concurrently using one or more embodiments of the scaffolds.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present concepts in a simplified form as a prelude to the more detailed description that is presented later.

### III. Scaffolds

Scaffolds are disclosed herein. In some embodiments, the scaffolds are produced by the methods disclosed herein. Disclosed herein are scaffolds comprising semi-stable fibers of a polymer oriented in a grid pattern. The semi-stable fibers may vary between bent (or chaotically bent) and substantially straight and form the hybrid fibrous scaffold. The scaffold may be layered in a grid pattern. The grid pattern may be arranged such that each layer is stacked in the same X- and Y-axis as the layer below or above it.

In some embodiments, the scaffold may comprise one or more therapeutic agents, such as two, three, four, five, six or more therapeutic agents. The one or more therapeutic agents may be loaded with the scaffold. In embodiments, the hybrid fibrous scaffold can be sprayed, dipped, coated, or otherwise covered in a therapeutic agent. The therapeutic agent can be incorporated within the polymer-containing solution prior to, during, or after formation of the scaffold. In embodiments, a first therapeutic agent can be incorporated within the polymer-containing solution and a second therapeutic agent can be sprayed, dipped, coated, or otherwise adhered to the surface of the scaffold.

The amount of loaded therapeutic agent may be varied. In some embodiments, the concentration of the loaded therapeutic agent within the polymer-containing solution is less than 1 mg/mL, about 1 mg/mL, about 1.5 mg/mL, about 2 mg/mL, about 2.5 mg/mL, about 3 mg/mL, or greater than 3 mg/mL. In some embodiments, the amount of loaded therapeutic agent is from 0.1 mg/mL to 25 mg/mL or 0.5 mg/mL to 15 mg/mL, or any subrange or subvalue thereof.

The one or more therapeutic agents can comprise a pharmaceutical composition, a biological fluid, or any other therapeutic compound known by one of skill in the art. The one or more therapeutic agents may comprise one or more of: an anticancer therapeutic, an antiviral, an antibiotic, an antihistamine, an antifungal agent, an anti-parasitic, an anti-inflammatory agent, an anesthetic, an analgesic, a growth factor, an antibody, peptide, a cytokine, a chemokine, an immunomodulatory agent, a radioactive composition, a clotting aid, a steroid, platelet-rich plasma, or a combination thereof.

The scaffold may release the one or more therapeutic agents over a duration of time. The release may be an extended release. An “extended” release includes continuous, sustained, and/or intermittent release of variable doses over a duration of time (e.g., the amount of the dose may be constant or changing through the release). The duration of the release and the amount of dose released may be varied. For example, the duration of the extended release may be less than 5 weeks to greater than 30 weeks. In some embodiments, the duration may range from about 5 weeks to about 30 weeks, from about 10 weeks to about 20 weeks, from about 12 weeks to about 18 weeks, or from about 14 weeks to about 17 weeks. In a specific embodiment, the therapeutic agent may extendedly release for about 17 weeks. The dose may be less than 5 μg per week to greater than 500 μg per week. In some embodiments, the dose may range from about 2 μg per week to greater than 100 μg per week, from about 4 μg per week to about 50 μg per week, from about 6 per week to about 30 μg per week, from about 8 μg per week to about 25 μg per week, and from about 10 μg per week to 20 μg per week.

The scaffold disclosed herein may further comprise one or more biocompatible materials. The biocompatible material may be any material selected to produce a desired and/or beneficial result. For example, the scaffold may include a component of the extracellular matrix. In some embodiments, the scaffold may include nonstructural elements, for example, growth factors, proteoglycans, or other biomolecules. In some embodiments, the biocompatible material comprises platelet-rich plasma. These and other nonstructural elements may be included to influence cell behavior, prevent infection, or otherwise conditions that improve the suitability of the materials for particular uses. The scaffold may comprise cells, such as living cells, including fibroblasts, platelets, stem cells, and the like. Further examples of biocompatible materials that may be added to the scaffold are water, saline, Hank's Balanced Salt Solution (HBSS), salts, buffers (such as HEPES or PBS), and serums (such as BSA).

### IV. Uses

The hybrid fibrous scaffolds of the present disclosure find use in therapeutic methods or uses, such as implant material scaffolds for cardiovascular tissue regeneration, musculoskeletal tissue regeneration, cancer therapies, immunotherapies such as a scaffold for creating an artificial thymus to implementation, or preventing or treating disease or injury in a subject (e.g., an animal or a human). The scaffolds may be administered (e.g., directly or indirectly) to a target tissue or organ (such as one that is damaged or diseased) to establish functional connections to regenerate or treat the target tissue or organ. The scaffolds may be applied to contact (e.g., cover, surround, or fill) a bone or tissue defect, a wound, or a surgical site. In some embodiments, the scaffold may be used as a vascular graft to bypass, or redirect, blood flow from one area to another via connecting blood vessels, such as to bypass a diseased artery. Indeed, the scaffolds of the disclosure find use as comprised in a vascular graft.

In one embodiment, the scaffolds enhance tissue regeneration, such as soft tissue (e.g., cardiovascular tissue or cells). Methods for repairing damaged tissue or organs may be carried out either in vitro, in vivo, or ex vivo.

In another embodiment, the disclosure provides a method of promoting endothelialization in a subject, the method comprising implant the scaffolds as a vascular graft in the subject.

The foregoing methods are not exclusive. One or more methods may be employed concurrently using one or more embodiments of the scaffold.

### V. Working Example 1: A Method of Making a Scaffold Comprised of PDO and Having Highly Aligned Scaffold Grids with a 3D Printer

Scaffolds made with the method of electrospinning of the present disclosure were studied. This study assessed the tensile strength, percent elongation, yield stress, yield elongation, and Young's modulus effect of said scaffolds.

### INTRODUCTION

Traditional Electrospinning (TES) is a popular method of creating fibrous scaffolds that mimic the extracellular matrix [1]. In this process, a polymer dissolved in solution is charged and paired with a counter electrode. The subsequent electric field exerts a force on the polymer solution drawing in towards the counter electrode in a conical shape termed a Taylor cone, and if the force of the electric field exceeds the surface tension of the polymer solution, a liquid jet is extruded and accelerated towards the counter electrode [2]. In the air gap, the solvent evaporates to form a solid fiber which incurs numerous bending instabilities that result in the fiber being randomly deposited on the collecting surface as a non-woven, fibrous material [3,4].

While TES allows for highly tailorable fiber diameters, there are limitations on the extent of tailorable pore sizes and fiber geometry. Indeed, there is an intrinsic proportional link in TES (non-woven structures) between fiber diameter and pore size, which presents a problem for the creation of physiologically relevant fiber diameters while maintaining sufficiently large pore sizes for cell and capillary ingrowth [5-7]. Furthermore, TES manufactured fiber geometries are limited from random to loose-aligned, depending on the rotational speed used on the grounded collector [8]. These limitations present significant design challenges, as native tissues have complex geometries and can exhibit tissue-engineered extracellular matrices (ECMs) with highly ordered orientations compounded with numerous in vitro studies demonstrating ECM alignment is a critical parameter in dictating cell alignment and behavior [9-11].

Towards these limitations, the sub-technique of TES termed near-field electrospinning (NFES) takes the expansive air gap distance and shortens it to a few millimeters before any bending instabilities can occur [12,13]. This reduced air gap is then paired with precise relative motion between the charged polymer capillary and the grounded collector. When the relative velocity is tuned to match the flow of polymer, then an extruded fiber can be directly written onto a substrate. Therefore, this technique allows for further scaffold tailorability, as individual fibers can be programmed with great precision to form 3D scaffolds that are laid down layer-by-layer [14]. To date, the vast majority of published fiber writing geometries feature perfectly stacked grids or triangles. While these architectures are perfect in form, the resulting stacked fibers appear as contiguous structures as opposed to fibrous constructs from a cell interaction perspective [15]. Furthermore, these superstructures frequently feature expansive grid pores that cells cannot interact with and subsequently either pass through or adhere to the stacked solid walls [16,17].

In this study, an architecture of highly-aligned grid structures with low-density random fiber infill for scaffold tailorability independent of fiber diameter is created having an air gap existence such that a semi-stable fiber is written in a grid pattern. Indeed, it was demonstrated that by altering this grid size, the surface pore size, as well as effective objective transit size, can be tailored. Furthermore, this scaffold tailorability can modulate the mechanical properties of ultimate tensile strength, yield strength, Young's modulus, yield strength, and yield elongation. Lastly, the neutrophil innate immune response of DNA extrusion to form neutrophil extracellular traps (NETs) can be further attenuated by these highly porous constructs.

### Experimental Methods

Scaffold Fabrication. A consumer 3D printer (Prusa 12″ Basic Pegasus, Maker Farm, South Jordan, Utah, USA) was modified by replacing the filament extrusion print head to accommodate an NFES print head, modified from our previous published NFES work [14]. The NFES print apparatus comprised a remote head syringe pump (Legato 130, KD Scientific Inc, Holliston, Mass., USA) secured in a custom-designed holder. The syringe pump held a polypropylene syringe and a blunt Luer-lock needle charged by a DC voltage source (HV050REG(+), Information Unlimited, Amherst, N.H., USA). The print head was able to translate in the X-axis and Z-axis, while the grounded collector translated in the Y-axis. The translational path was written in G-code and sent to the 3D printer using the 3D print software Repetier (Hot-World GmbH and Co. KG, Willich, Germany).

Polydioxanone (DIOXOMAXX 100, Inherent viscosity 2.13 dL/g, Bezwada Biomedical, LLC, Hillsborough, N.J., USA) solutions were dissolved overnight in 1,1,1,3,3,3-hexafluoro-2-propanol (HFP) (Oakwood Products, Inc., Estill, S. C., USA) at a concentration of 112 mg/mL for all NFES scaffolds. The air gap was set at 3 mm, an applied voltage of 1.6 kV, a polymer flow rate of 25 μL/hr through a 23-gauge, 2-inch blunt needle, and print head translation velocity of 80 mm/s to produce fibers with a target diameter range of 1-2 μm. Fibers were deposited on a 6 inch×6 inch tight-tolerance, electrically grounded aluminum plate (Cat No. 3511T151, McMaster-Carr, Elmhurst, Ill., USA) on the NFES print bed in a relative humidity environment of 45%— 55%. After the initial fiber extrusion, the NFES print head was translated at 10 mm/s for 60 s to allow for fiber deposition to stabilize before the main G-code program was executed.

Grids were programmed with X- and Y-grid spacing of 200×200 μm (NFES 200²), 300×300 μm (NFES 300²), 400×400 μm (NFES 400²), and 500×500 μm (NFES 500²), Table 1. Scaffold layer counts were chosen to give all resulting constructs the same average thickness. TES scaffolds with fiber diameters of 1-2 μm were fabricated with a PDO concentration of 140 mg/mL at an applied voltage of +25 kV, 17.8 cm air gap, a polymer flow rate of 4 mL/h, through an 18 gauge, 2 inch length needle. TES scaffolds with fiber diameters of 0.3 μm-0.4 μm were fabricated with a PDO concentration of 55 mg/mL at an applied voltage of +28 kV, 28 cm air gap, a polymer flow 0.65 mL/h, through a 22.5 gauge, 1 inch length needle. TES fibers were collected on a grounded, stainless steel mandrel (rectangular dimensions: 20×75×5 mm³) rotating at 1250 rpm and translating 6.5 cm/s over a distance of 13 cm.

TABLE 1
NFES scaffold and layer processing parameters

| | |
|---|---|
| Effective Pore Size and | Neutrophil |
| Mechanical Testing | Interactions |

| Grid Size | Scaffold | Layer | Scaffold | Layer |
|---|---|---|---|---|
| (μm) | Size (mm) | Count | Size (mm) | Count |
| 200 × 200 | 84 × 22 | 250 | 60 × 60 | 100 |
| 300 × 300 | 84 × 22 | 300 | 60 × 60 | 200 |
| 400 × 400 | 84 × 22 | 400 | 60 × 60 | 250 |
| 500 × 500 | 84 × 22 | 500 | 60 × 60 | 300 |

After production, scaffolds were placed in a room-temperature vacuum chamber (ISOTEMP® Vacuum Oven Model 281A, Fisher Scientific, Waltham, Mass., USA) for 10 minutes at a minimum of 70 kPa below atmospheric pressure to remove any remaining solvent. Scaffolds were then stored in a desiccant chamber until use.

A small sample was dissected from all fabricated scaffolds for imaging. Samples were sputter-coated in an argon atmosphere with 5.0 nm of 60:40 gold-palladium and visualized using a scanning electron microscope (Nova Nano 650 FEG, FEI Co., Hillsboro, Oreg., USA) with the field emission gun at +20 kV, a spot size of 3, and 5 mm working distance. Fiber diameters and scaffold gride sizes were measured from acquired images using the software Fibraquant v1.3.149 (nanoScaffold Technologies, Chapel Hill, N.C., USA) and FIJI v2.1.0/1.53c. The software was calibrated from micrograph scale bars and fiber diameter averages were calculated from a minimum of 60 semi-automated random measurements per image while grid sizes were calculated from a minimum of 15 manual measurements. All NFES and TES equal scaffolds used had measured fiber diameters with an average between 1 μm-2 μm, while small fiber diameter TES scaffolds for in vitro work had averages between 0.3 μm-0.4 μm. Scaffolds for effective pore size and mechanical testing had thickness between 0.09 mm-0.12 mm.

Scanning Electron Micrograph Pore Size Analysis. Surface pore sizes were determined from low magnification micrographs. A java macro was written in FIJI v2.1.0/1.53c to auto-threshold the micrographs using Otsu's methods for discerning foreground versus background, followed by applying a watershed algorithm. The FIJI function Analyze Particles was used to measure pore diameters of the resulting top layer using the parameters of particle size: 10-250,000 μm², 0-1 circularity, excluded edges, and holes weren't included. A minimum of (n=4) scaffolds was used to determine the average surface pore size.

Effective Pore Size Analysis. Fluorescent microsphere filtration was used to ascertain the effective restriction size of an object transiting the NFES and TES scaffolds [18]. Green fluorescent microspheres with 9.9 μm and 97 μm diameters (Cat No. G100, 35-11, Fisher Scientific, Waltham, Mass., USA) were diluted to working concentrations in deionized (DI) water as well as stored and handled in dark conditions per manufacture instructions. Scaffolds (n=5) were punched out into 8 mm disks using a medical biopsy punch (Acu-Punch, 8.0 mm, Acuderm Inc., Fort Lauderdale, Fla., USA), pre-hydrated in DI water, and placed inside a custom machined apparatus designed to securely hold thin membranes for filtration evaluation through a 6 mm diameter hole. A polypropylene syringe loaded with 1.6 mL of either 500 microspheres/mL of 97 μm or 50,000 microspheres/mL of 9.9 μm spheres and was loaded into an upright syringe pump (NE-300 Just Infusion″, New Era Pump Systems, Inc., Farmingdale, N.Y., USA) with a flow rate of 0.25 mL/min. One milliliter of microsphere solution was passed through the scaffolds while the remaining volume accounted for apparatus dead space. In between samples, the apparatus was flushed with 10 mL of DI water to remove any adherent microspheres.

NFES 200², 300², 400², and 500² grid sizes were evaluated along with TES scaffolds of equivalent fiber diameters. The absence of a membrane served as a positive control and filter paper with 7-8 μm pores served as a negative control (Cat No. 09-803-6F, Fisher Scientific, Waltham, Mass., USA). The filtrate was transferred to a black with clear bottom 96 well plate and measured in duplicate using a spectrophotometer (SpectraMax i3, Molecular Devices, San Jose, Calif., USA), compared to a standard curve of known microsphere counts. Each well area was scanned in its entirety with 37 measurements from a 1 mm beamwidth using an excitation of 468 nm and an emission of 508 nm. The calculated microsphere count was then normalized to the area of flow.

Mechanical Characterization. Scaffolds were mechanically evaluated using a uniaxial testing frame in tension, equipped with a 25 lbf load cell (Model SM-25-294, TestResources, Inc, Shakopee, Minn., USA) until failure. Methodology adhered closely to ASTM D1708-18 as scaffolds were punched (n=8) out using a “dog bone” punch adhering to a Type V specimen as determined by ASTM D638-14, with a gauge length of 7.5 mm and thickness of 2.93 mm [19,20]. Over macroscopic distances, TES scaffolds are homogenous and isotropic while NFES grid scaffolds are homogenous and anisotropic. Therefore, NFES scaffolds were punched out parallel to the grid structure as well as 45° to the grid axis. Before testing, scaffolds were submerged in phosphate-buffered saline (PBS) and placed in a 37° C. incubator for a minimum of 90 minutes to equilibrate to physiological temperatures. Scaffolds were immediately and securely fastened into knurled grips and separated at a rate of 10 mm/minutes until failure. Material elongation at failure, ultimate tensile stress, Young's Modulus, yield elongation, and yield stress were reported.

### Human Neutrophil NETosis Response

Cell Culture. Neutrophils were isolated from heparinized whole blood obtained via venipuncture following previously published protocols [21-23]. Blood was donated from Tennessee Blood Services which was donated from healthy donors. Since purchased or donated samples are not traceable back to the donor, it does not qualify as human subjects research as determined by the University of Memphis Institutional Review Board on Nov. 22, 2016. Cells were collected at room temperature from the supernatant after Isolymph sedimentation and from the pellet of an Isolymph density gradient (Cat No. 50-300-403, Fisher Scientific, Waltham, Mass., USA) under endotoxin-free conditions. The contaminating erythrocytes were lysed in ice-cold, hypotonic 0.2% sodium chloride solution for 30 s, followed by the restoration of physiological tonicity by the addition of hypertonic 1.6% sodium chloride solution. This process resulted in neutrophil viability >98% as assessed by trypan blue dye exclusion. The isolated neutrophils were washed once in Hank's buffered salt solution (HBSS) and resuspended at a density of 1×10⁶ cells/mL in HBSS with 0.2% autologous human serum, and 10 mM HEPES.

NFES and TES scaffolds were UV sterilized (Cat No. EN-280L 1090 μW/cm² at 15 cm, SPECTROLINE®, Westbury, N.Y., USA) for 10 minutes at a distance of 10 cm from the source on each side and placed in a 96-well plate before seeding. Scaffolds were hydrated with 50 μL of HBSS with 0.2% autologous serum and 10 mM HEPES. Scaffolds were seeded with 100 μL of 1×10⁶ cells/mL cell suspension (100,000 cells/well) and cultured for 3 and 6 hours under standard culture conditions of 37° C. and 5% CO₂.

After incubation, cells were placed on ice to inhibit further stimulation. The supernatant was removed from the cell-laden scaffolds, and the cells were fixed in their wells on the scaffolds with 10% buffered formalin. Cells were stained with 4′,6-diamidino-2-phenylindole (DAPI) (NucBlue™ Fixed Cell Stain ReadyProbes™, Molecular Probes, Eugene, Oreg., USA) per manufacture concentration for 5 minutes followed by 5 μM SYTOX Orange (Cat No. 511368, Fisher Scientific, Waltham, Mass., USA) for 15 minutes. Cells were then permeabilized with 0.1% Triton X-100 for 10 minutes at room temperature and stained with a phalloidin stain per manufacture concentration for 30 min (ActinGreen™ 488 ReadyProbes™, Invitrogen, Carlsbad, Calif., USA). Scaffolds were stored in 96 well plates submerged in PBS at 4° C. and were imaged within 72 hours.

Microscopy and Analysis. Scaffolds were placed on glass slides, mounted with PBS, and covered with a glass cover slide. Fluorescently stained cells on scaffolds were imaged with an Olympus BX 63 fluorescent microscope (Olympus Corporation, Center Valley, Pa., USA) with an Olympus DP80 CCD camera using a 20× objective with a 0.6 mm working distance. Images were acquired in cell Sens Dimensions v2.3 and saved as lossless VSI and tiff file formats. Exposure was set based on being below the saturation limit of the CCD camera for the TES_{0.3-0.4 μm} material reference positive control. Serial images of scaffolds were acquired over a range of focal planes to form a 25 μm thick Z-stack image for 3 random locations. Step size was determined by the Nyquist frequency of the limiting wavelength of the DAPI filter. Olympus's cellSens TruSight deconvolution software was used to further process the Z-stack images using the Constrained Iterative Filter for 3 iterations followed by forming a 2D extended focal image (EFI) from the deconvolved Z-stacks. Images were analyzed using a previously published MATLAB vR2020a program to count the percent area covered by NETs [21-23].

Statistics. All statistical analysis was performed in Prism 8 v8.2.1 (GraphPad Software Inc., San Diego, Calif., USA). Differences were tested between groups using ANOVA with Holm-Sidak's multiple comparisons at a significance of p<0.05. Grid sizes were compared to their theoretical programmed value using a one-sample t-test at a significance of p<0.05. All data were reported as mean±standard deviation.

### Results

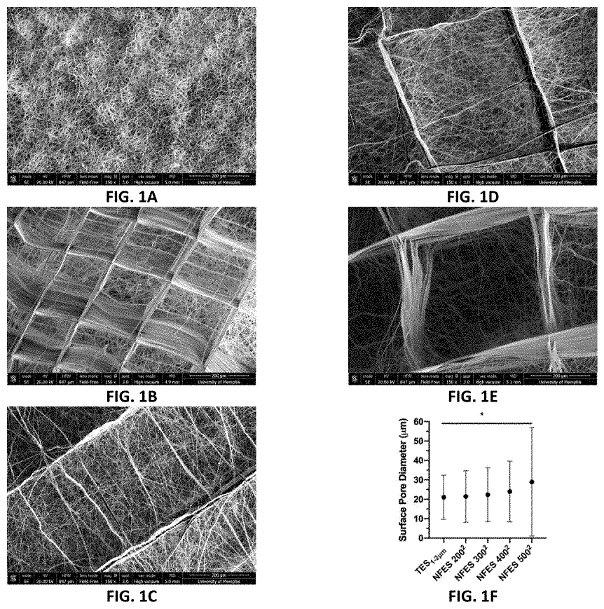

Scaffold Fabrication and SEM Analysis. The TES and NFES manufacturing process resulted in regular PDO fibers free of beading with average diameters ranging between 1-2 μm, FIGS. 1A-E. Qualitatively, the highly aligned grid structure was present in all NFES scaffolds with a random infill of fibers, and quantitatively there was no significant difference between the four theoretical programmed grid sizes and the manufactured grid size (p>0.05). For surface pore size, the fibrous infill resulted in a small but statistically significant difference between the TES and NFES scaffolds with means ranging from TES with 21.0±11.4 μm to NFES 500² with 28.9±27.9 μm, FIG. 1F. Additionally, there was a statistically significant difference between each of the scaffolds suggesting that the increase in programmed grid size correlates with surface pore size. This result is more evident when evaluating the 90% percentile scaffold pore sizes as the data for TES and NFES 200² scaffolds showed 35.8 μm and 36.9 μm, respectively. NFES 500² scaffolds had a 90% percentile pore size of 53.8 μm, which together suggests that the NFES grid scaffolds are forming larger average pores with a few substantially large pores at the surface. Therefore, these data suggest that the present disclosure permits architecture pore size to be an independently adjusted property from fiber size.

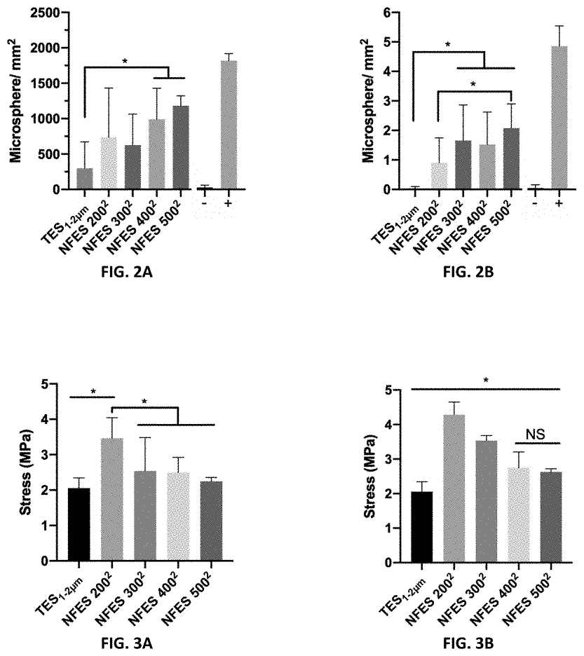

Effective Pore Size Analysis. To evaluate the object transit size through the thickness of the TES and NFES scaffolds, fluorescent microspheres were filtered through hydrated TES and NFES scaffolds. This 3D effective pore size was evaluated for 9.9 μm spheres, on the order of magnitude of cells, and 97 μm spheres on the magnitude of capillaries with supporting pericytes [5,24,25]. For the 9.9 μm spheres, both the NFES 400² and 500² scaffolds with permeabilities of 737±697 microspheres/mm² and 627±436 microspheres/mm², respectively, were significantly more permeable by a factor of 3 compared to the TES scaffold, FIG. 2A. Within the NFES scaffolds, there was no statistical difference, but a linear increase in sphere permeability per unit area was observed. The data show for the filtration of 97 μm microspheres that there was a significant difference between the TES scaffold, which was virtually impermeable, and NFES 300² through 500², FIG. 2B. Furthermore, there was a significant difference within the NFES scaffolds as the 500² was more than twice as permeable to the 97 μm microspheres with 2±0.82 microspheres/mm² compared to NFES 200² as well as a near-linear trend being observed. Taken together these data suggest that NFES object transit permeability can be tailored as a function of scaffold programming independent of fiber diameter.

Mechanical Characterization. NFES grid scaffolds were mechanically evaluated in their principal and 45° axis along with TES scaffolds of the same 1 μm-2 μm fiber diameter. In the principal axis, NFES 200² scaffolds had a significantly greater UTS of 1.5-fold compared to the 2.1±0.3 MPa of TES scaffold as well as significantly greater than all other NFES scaffolds, FIG. 3A. In the 45° axis, all comparisons were significantly different except for NFES 400² and 500², FIG. 3B. The TES scaffold had the lowest UTS at less than half the value of 4.3±0.4 MPa for NFES 200². Within the NFES there was a linear trend observed with larger grid sizes resulting in a reduction in UTS.

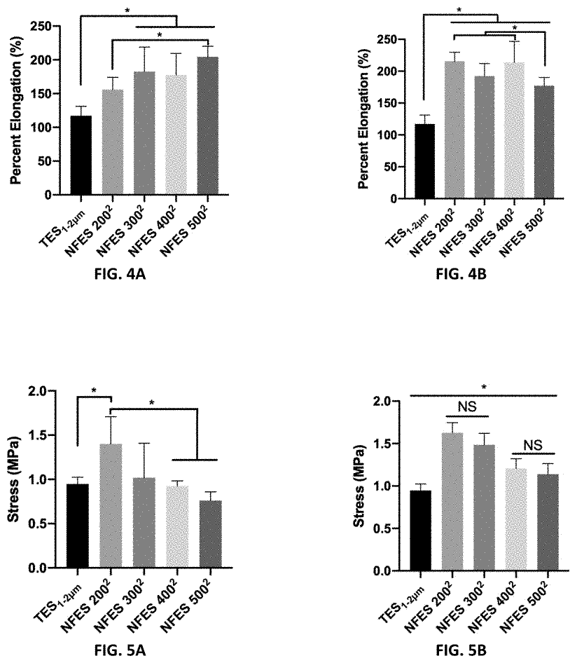

Percent elongation at failure in the principal axis showed a significantly lower difference between the TES scaffold with 117±14% and all other NFES scaffolds except NFES 200², FIG. 4A. Compared to TES, NFES 500² scaffolds were 2-fold more ductile at failure. Within the NFES scaffolds, a linear trend was observed as increased grid size resulted in greater elongation at failure. Furthermore, there was a significant increase of 50% elongation between NFES 200² and NFES 500². In the 45° axis, the TES scaffold was significantly less ductile by a minimum of 60% elongation compared to every NFES scaffold, FIG. 4B. Within the NFES scaffolds, NFES 500² with 177±13% was significantly less than NFES 200² and 400². This result is a reverse of the trend seen in the principal axis where NFES 500² was the most ductile.

The data for yield stress in the principal axis showed that NFES 200² had the greatest stress at yield of 1.4±0.3 MPa and was the only NFES scaffold that was significantly greater than the TES scaffold by 1.45-fold, FIG. 5A. Within the NFES scaffolds, NFES 200² was significantly greater than both NFES 400² and 500² by 1.5 to 2-fold. Furthermore, a linear trend was observed with the closest scaffold grids resulting in the greatest stress at yield. In the 45° axis, all comparisons were significant except for NFES 200² and 300² as well as NFES 400² and 500², FIG. 5B. The TES scaffold had the lowest yield stress of 0.9±0.1 while NFES 200² had the greatest yield stress of 1.7-fold that of TES. Lastly, there was a prominent linear trend in the 45° axis with increasing the grid spacing resulting in a reduction in yield stress.

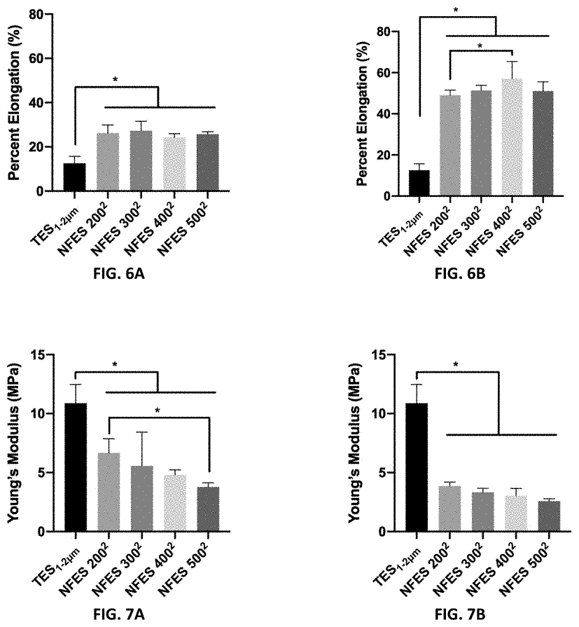

Elongation at the yielding point in the principal axis showed a significant increase in all NFES scaffolds by a factor of 2 compared to 12.6±3.2% for the TES scaffold, FIG. 6A. There were no significant differences or trends observed within the NFES scaffolds. Elongation at yield within the 45° axis also showed a significant increase in all NFES scaffolds of 4-fold increase compared to the TES scaffold, FIG. 6B. Within the NFES grid scaffolds, NFES 400² was significantly greater than NFES 200², but despite this difference, there was no observed trend in the NFES scaffolds as was in the principal axis.

In both axes, the TES scaffold had a significantly greater Young's modulus of 10.9±1.6 MPa compared to all NFES scaffolds. In the principal axis within NFES scaffolds, NFES 200² had the stiffest modulus which was approximately 60% of the TES scaffolds and was significantly greater than NFES 500². Furthermore, there was a linear trend in Young's modulus observed as larger spaced grids resulted in a reduction in stiffness. In the 45° axis, TES scaffolds had a modulus approximately three-fold greater than all NFES scaffolds. Also, there were no significant differences within the NFES scaffolds, but a linearly decreasing trend in Young's modulus was observed for increasing grid size with NFES 500² having the lowest modulus of 2.6±0.2 MPa. Taken together these data suggest that NFES scaffold's mechanical properties can be tailored as a function of programming and thus is independent of fiber diameter. Compared to TES scaffolds of the same fiber diameter, NFES has a lower modulus while having a greater UTS, elongation at failure, yield stress, and yield elongation within the geometries evaluated.

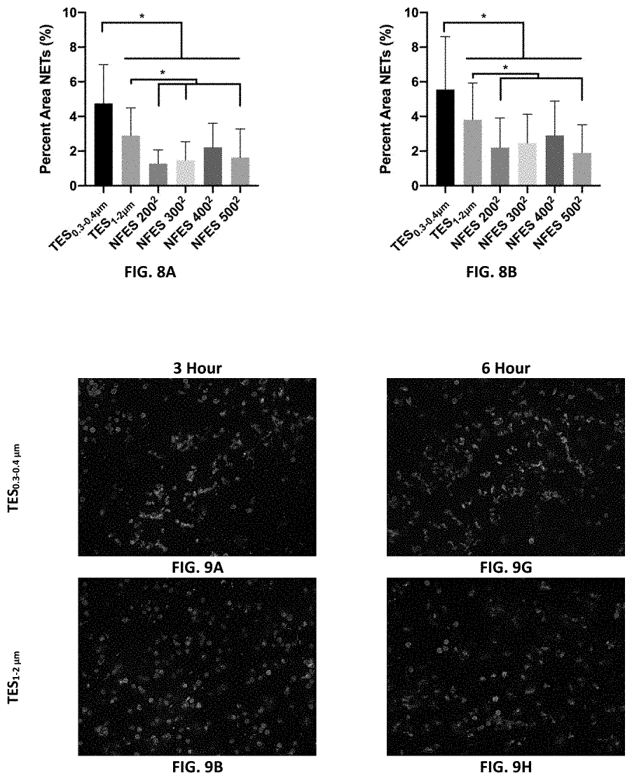

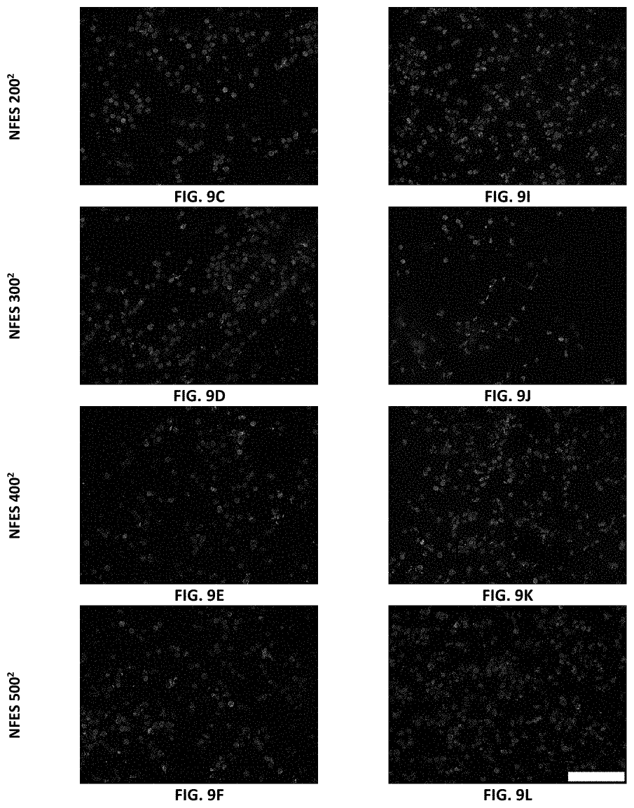

Human Neutrophil NETosis Response. Our previously published results with human neutrophils interacting with PDO architecture showed that smaller diameter fibers, 0.3-0.4 μm, result in approximately twice the area covered by NETs compared to a larger diameter, 1-2 μm [21-23]. These differences are seen in the data as material reference controls, FIGS. 8A and 8B. To visualize scaffold interacting cells on these highly 3D constructs, 25 μm Z-stacks were acquired followed by quantitative deconvolution and extended focal projection processing to result in a 2D in-focus image, FIG. 9A-L. Cell nuclei count was compared for all NFES scaffolds compared to TES_{1-2 μm} to insure no under-sampling resulting from cells below the imaging thickness. At 3 hours the NFES 200² and 300² scaffolds had statistically lower nuclei counts compared to TES_{1-2 μm}, but at 6 hours there were no differences between all NFES scaffolds and TES_{1-2 μm}. These results suggest that as the lower nuclei counts were present on the smaller pore size scaffolds with no difference compared to the larger pore size scaffolds, that these differences can be attributed to seeding and imaging variation. Combined with no difference at 6 hours suggests that imaging of these highly porous and 3D scaffolds can be quantitatively compared.

At 3 hours, there was a significant reduction in NETs of approximately 50% in NFES 200², 300², and 500² compared to the 4.8±2.2% area coverage of TES_{1-2 μm}. Within the NFES scaffolds, there was no trend or significant difference observed in the percent area covered. At 6 hours, there was a significant reduction in NETs of again approximately 45-50% in NFES 200² and 500² scaffolds compared to the 3.8±2.1% area coverage of TES_{1-2 μm} scaffolds. Within the NFES scaffolds at 6 hours there was no trend or significant difference observed in the percent area covered.

The deconvolved extended focus images resulted in highly in-focus images for accurate analysis of the Z-depth varied NFES scaffolds, FIGS. 9A-9L. At 3 hours, the TES_{0.3-0.4 μm} scaffolds show few intact nuclei, as seen by the blue channel DAPI stain, and prominent coverage of NETs stained with Sytox Orange in the red channel. TES_{1-2 μm} and NFES scaffolds all have few NETs present and intact cell nuclei at 3 hours. At 6 hours there is a further increase in these trends as seen with greater coverage of NETs on scaffolds. TES_{0.3-0.4 μm} scaffolds show extensive coverage with few intact nuclei. TES_{1-2 μm} and NFES scaffolds showed fewer NETs coverage with NFES 500² scaffolds showing mostly compromised nuclei with few extrude NETs.

Taken together the quantitative data and representative images suggest that NFES scaffolds pore size architecture results in a reduction in human neutrophil NETs at 3 and 6 hours compared to TES_{0.3-0.4 μm} scaffolds and a lesser degree TES_{1-2 μm}. There were no discernable trends within the NFES scaffolds, suggesting that fiber densities beyond a certain threshold don't further attenuate NET release.

### DISCUSSION

While TES is an excellent manufacturing technique to create highly porous, interconnected scaffolds that resemble the extracellular matrix, unless specialized setups such as air impedance or porogens are used, an electrospun scaffold's pore and fiber diameter are intrinsically linked [1,26-29]. As fiber diameter increases, the subsequent reduction in surface area relative to volume results in less efficient packing and thus larger pores [6,7]. Tissue engineering design criteria typically dictates larger pores to facilitate cell and capillary ingrowth into the scaffold which, consequently, locks the range of fiber diameters that can be used as well as relegates the tailoring of mechanical properties to material composition and scaffold geometry.

NFES allows for further scaffold tailorability to directly program the placement of fibers to create highly ordered structures. Frequently, NFES structures that are published feature trivially large pore sizes with fibers perfectly stack one on top of the other. This perfect stacking results in a loss of fibrous qualities from a cell interaction perspective with the macrostructure. The hybrid semi-stable NFES strategy disclosed herein where the air gap results in a stochastic switch between directly writing a fiber and the chaotic bending extrusion of a fiber typically seen in TES was used. When the print head was programmed to translate in a stacking grid pattern, the resulting structure was highly aligned grid fibers that were intercalated with low density, random fibers. As the switching process can be considered random, increasing the grid size results in both a lower density of fibers in the center of each grid cell and intercalated into the grid structure as seen on SEM imaging as well as a lower density of “rebar-like” stacked fibers. Consequently, the degree of inefficiently packed fibers intercalated in the grid structure can increase the apparent scaffold thickness for a constant fiber diameter as seen by the increasing number of layers required to achieve the same scaffold thickness for increasing grid sizes in Table 1. Therefore, this technique decouples the association between fiber diameter and pore size, unlocking a new degree of tissue engineering scaffold tailorability, along with enabling the adjustment of stable and unstable in layer development on a layer-by-layer basis.

Surface pore size analysis of non-hydrated scaffolds fails to give any information on the effective, interconnected pore size of a scaffold. While many modalities exist to measure pore sizes such as volume/density/displacement measurements, liquid intrusion, and micro CT, these techniques range from being unable to measure the interconnected size of pores in a structure (which is essential for the travel of an object), use of toxic materials such as nonwetting mercury, or the use of highly expensive equipment relegated to core facilities [30]. Fluorescent microsphere permeability is an inexpensive method of determining if an object of a given size can traverse a porous membrane. While exact pore sizes vary in the literature, 9.9 and 97 μm microspheres were chosen to give a biologically relevant range of effective object size permeability. Despite the 1-2 μm fibers, which are in the upper range of fiber sizes for this technique, TES scaffold's relatively large pore sizes were moderately permeable to the 9.9 μm microsphere and were impermeable to the 97 μm microspheres. This was in contrast to all of the NFES grid sizes being linearly permeable to the 97 μm microspheres at the same fiber diameter. Furthermore, the range of explored grid sizes was not bounded as it is possible to further increase as well as decrease the size based on design criteria needs.

Despite the increase in effective permeability, NFES scaffolds mechanical properties were not adversely attenuated. The Young's modulus of the bulk NFES material was both tailorable and systematically lower than TES. Furthermore, the NFES scaffolds were tailorable to have greater UTS, percent elongation, yield stress, and yield elongation compared to TES scaffolds of the same fiber diameter. These improvements are primarily due to the reinforcing grid structure in the scaffolds working in concert. Attenuating the architecture of “rebar-like” grids per unit area results in a tailorable range of mechanical properties which consequently also attenuates average pore size.

The general consensuses in the literature agree that larger, interconnected pores are a favorable tissue engineering strategy [31,32]. Specifically, within the innate immune system, our previous TES work has demonstrated that larger fiber diameters/pore sizes result in the regenerative M2 phenotype in macrophages [33]. Furthermore, in our previous neutrophil work, large diameter, 1 μm-2 μm, PDO fibers with subsequently larger pore sizes resulted in a reduction in NETs released onto the scaffolds compared to small diameter, 0.3 μm-0.4 μm, fibers and associated pore sizes [21,22]. The attenuation of excessive NET release is favorable as NETs are inflammatory and highly thrombogenic and this presents the question of which elements cause the reduction of NETs: fiber diameter or pore size [34-36]. While the NFES technique to produce a scaffold comprised of 0.3 μm-0.4 μm to fibers is not currently possible, it was shown that a further increase in pore size does further attenuate the formation of NETs. This decrease was limit and no linear trend was observed beyond the initial decrease. It is believed that this observation was due to that a neutrophil's size can only contact a finite space and a decrease in density beyond this range cannot be detected.

### CONCLUSION

NFES allows for unprecedented control over PDO scaffold creation. This control was further leveraged to create a hybrid geometry of highly ordered, stacked fibers and low-density random fiber infill. As a result, biologically relevant pore sizes and mechanical properties can be tailored as a function of programming, independent of fiber diameter. This increased pore size also has a beneficial attenuation of the inflammatory and highly thrombogenic phenomenon of NET release on neutrophil interacting scaffolds. Future work necessitates decreasing the fiber diameter range of NFES to further elucidate observations of fiber diameter and pore size. It is believed that NFES of PDO as well as our presently disclosed architecture present an advancement for numerous biomedical applications in areas such as vascular, neural, and wound bed tissue engineering.

### REFERENCES

1. Greiner, A.; Wendorff, J. H. Electrospinning: a fascinating method for the preparation of ultrathin fibers. Angew Chem Int Ed Engl 2007, 46, 5670-5703, doi:10.1002/anie.200604646.
2. Taylor, G. Disintegration of Water Drops in an Electric Field. Proceedings of the Royal Society of London. Series A, Mathematical and Physical 1964, 280, 383-397, doi:10.1098/rspa.1964.0151.
3. Hohman, M. M.; Shin, M.; Rutledge, G.; Brenner, M. P. Electrospinning and electrically forced jets. I. Stability theory. Physics of Fluids 2001, 13, 2201-2220, doi:10.1063/1.1383791.
4. Doshi, J.; Reneker, D. H. Electrospinning Process and Apllications of Electrospun Fibers. Journal of Electrostatics 1995, 35, 151-160, doi:10.1016/0304-3886(95)00041-8.
5. Walthers, C. M.; Nazemi, A. K.; Patel, S. L.; Wu, B. M.; Dunn, J. C. The effect of scaffold macroporosity on angiogenesis and cell survival in tissue-engineered smooth muscle. Biomaterials 2014, 35, 5129-5137, doi: 10.1016/j.biomaterials.2014.03.025.
6. Pham, Q. P.; Sharma, U.; Mikos, A. G. Electrospun poly(epsilon-caprolactone) microfiber and multilayer nanofiber/microfiber scaffolds: characterization of scaffolds and measurement of cellular infiltration. Biomacromolecules 2006, 7, 2796-2805, doi:10.1021/bm060680j.
7. Szentivanyi, A.; Chakradeo, T.; Zernetsch, H.; Glasmacher, B. Electrospun cellular microenvironments: Understanding controlled release and scaffold structure. Adv Drug Deliv Rev 2011, 63, 209-220, doi:10.1016/j.addr.2010.12.002.
8. Murugan, R.; Ramakrishna, S. Design strategies of tissue engineering scaffolds with controlled fiber orientation. Tissue Eng 2007, 13, 1845-1866, doi:10.1089/ten.2006.0078.
9. Wang, W. Y.; Pearson, A. T.; Kutys, M. L.; Choi, C. K.; Wozniak, M. A.; Baker, B. M.; Chen, C. S. Extracellular matrix alignment dictates the organization of focal adhesions and directs uniaxial cell migration. APL Bioeng 2018, 2, 046107, doi:10.1063/1.5052239.
10. Rhodin, J. Architecture of the vessel wall. In Handbook of Physiology, Geiger, S., Bohr, D., Somlyo, A., Sparks Jr, H., Eds. American Physiological Society: Bethesda, Md., 1980; Vol. 2.
11. Timmins, L. H.; Wu, Q.; Yeh, A. T.; Moore, J. E., Jr.; Greenwald, S. E. Structural inhomogeneity and fiber orientation in the inner arterial media. Am J Physiol Heart Circ Physiol 2010, 298, H1537-1545, doi:10.1152/ajpheart.00891.2009.
12. Kameoka, J.; Orth, R.; Yang, Y.; Czaplewski, D.; Mathers, R.; Coates, G. W.; Craighead, H. G. A scanning tip electrospinning source for deposition of oriented nanofibres. Nanotechnology 2003, 14, 1124-1129, doi:10.1088/0957-4484/14/10/310.
13. Sun, D.; Chang, C.; Li, S.; Lin, L. Near-Field Electrospinning. Nano Lett 2006, 6, 839-842, doi:10.1021/n10602701.
14. King, W. E., III; Gillespie, Y.; Gilbert, K.; Bowlin, G. L. Characterization of Polydioxanone in Near-Field Electrospinning. Polymers (Basel) 2019, 12, doi:10.3390/polym12010001.
15. Blum, C.; Schlegelmilch, K.; Schilling, T.; Shridhar, A.; Rudert, M.; Jakob, F.; Dalton, P. D.; Blunk, T.; Flynn, L. E.; Groll, J. Extracellular Matrix-Modified Fiber Scaffolds as a Proadipogenic Mesenchymal Stromal Cell Delivery Platform. ACS Biomaterials Science & Engineering 2019, 5, 6655-6666, doi:10.1021/acsbiomaterials.9b00894.
16. Tylek, T.; Blum, C.; Hrynevich, A.; Schlegelmilch, K.; Schilling, T.; Dalton, P. D.; Groll, J. Precisely defined fiber scaffolds with 40 mum porosity induce elongation driven M2-like polarization of human macrophages. Biofabrication 2020, 12, 025007, doi:10.1088/1758-5090/ab5f4e.
17. Zhang, Z.; Jorgensen, M. L.; Wang, Z.; Amagat, J.; Wang, Y.; Li, Q.; Dong, M.; Chen, M. 3D anisotropic photocatalytic architectures as bioactive nerve guidance conduits for peripheral neural regeneration. Biomaterials 2020, 253, 120108, doi:10.1016/j.biomaterials.2020.120108.
18. Duke, S. Evaluating pore sizes of biological membranes with fluorescent microspheres. Particulate Science and Technology 1989, 7, doi:10.1080/02726358908906539.
19. ASTM D638-14; ASTM International: West Conshohocken, Pa., 2014.
20. ASTM D 1708-18; ASTM International: West Conshohocken, Pa., 2018.
21. Fetz, A. E.; Neeli, I.; Rodriguez, I. A.; Radic, M. Z.; Bowlin, G. L. Electrospun Template Architecture and Composition Regulate Neutrophil NETosis In Vitro and In Vivo. Tissue Engineering Part A 2017, 23, 1054-1063, doi:10.1089/ten.tea.2016.0452.
22. Fetz, A. E.; Neeli, I.; Buddington, K. K.; Read, R. W.; Smeltzer, M. P.; Radic, M. Z.; Bowlin, G. L. Localized Delivery of Cl-Amidine From Electrospun Polydioxanone Templates to Regulate Acute Neutrophil NETosis: A Preliminary Evaluation of the PAD4 Inhibitor for Tissue Engineering. Front Pharmacol 2018, 9, 289, doi:10.3389/fphar.2018.00289.
23. Minden-Birkenmaier, B. A.; Smith, R. A.; Radic, M. Z.; van der Merwe, M.; Bowlin, G. L. Manuka Honey Reduces NETosis on an Electrospun Template Within a Therapeutic Window. Polymers (Basel) 2020, 12, doi:10.3390/polym1206143 0.
24. Oliviero, O.; Ventre, M.; Netti, P. A. Functional porous hydrogels to study angiogenesis under the effect of controlled release of vascular endothelial growth factor. Acta Biomater 2012, 8, 3294-3301, doi:10.1016/j.actbio.2012.05.019.
25. Xiao, X.; Wang, W.; Liu, D.; Zhang, H.; Gao, P.; Geng, L.; Yuan, Y.; Lu, J.; Wang, Z. The promotion of angiogenesis induced by three-dimensional porous beta-tricalcium phosphate scaffold with different interconnection sizes via activation of PI3K/Akt pathways. Sci Rep 2015, 5, 9409, doi:10.1038/srep09409.
26. Huang, Z.-M.; Zhang, Y. Z.; Kotaki, M.; Ramakrishna, S. A review on polymer nanofibers by electrospinning and their applications in nanocomposites. Composites Science and Technology 2003, 63, 2223-2253, doi:10.1016/s0266-3538(03)00178-7.
27. Li, D.; Xia, Y. Electrospinning of Nanofibers: Reinventing the Wheel? Advanced Materials 2004, 16, 1151-1170, doi:10.1002/adma.200400719.
28. Selders, G. S.; Fetz, A. E.; Spear, S. L.; Bowlin, G. L. Fabrication and characterization of air-impedance electrospun polydioxanone templates. Electrospinning 2016, 1, doi:10.1515/esp-2016-0003.
29. Karageorgiou, V.; Kaplan, D. Porosity of 3D biomaterial scaffolds and osteogenesis. Biomaterials 2005, 26, 5474-5491, doi:10.1016/j.biomaterials.2005.02.002.
30. Bartos, M.; Suchy, T.; Foltan, R. Note on the use of different approaches to determine the pore sizes of tissue engineering scaffolds: what do we measure? Biomed Eng Online 2018, 17, 110, doi:10.1186/s12938-018-0543-z.
31. Pennel, T.; Zilla, P.; Bezuidenhout, D. Differentiating transmural from transanastomotic prosthetic graft endothelialization through an isolation loop-graft model. J Vasc Surg 2013, 58, 1053-1061, doi:10.1016/j.jvs.2012.11.093.
32. Pennel, T.; Bezuidenhout, D.; Koehne, J.; Davies, N. H.; Zilla, P. Transmural capillary ingrowth is essential for confluent vascular graft healing. Acta Biomater 2018, 65, 237-247, doi:10.1016/j.actbio.2017.10.038.
33. Garg, K.; Pullen, N. A.; Oskeritzian, C. A.; Ryan, J. J.; Bowlin, G. L. Macrophage functional polarization (M1/M2) in response to varying fiber and pore dimensions of electrospun scaffolds. Biomaterials 2013, 34, 4439-4451, doi:10.1016/j.biomaterials.2013.02.065.
34. Branzk, N.; Papayannopoulos, V. Molecular mechanisms regulating NETosis in infection and disease. Semin Immunopathol 2013, 35, 513-530, doi:10.1007/s00281-013-0384-6.
35. Schauer, C.; Janko, C.; Munoz, L. E.; Zhao, Y.; Kienhofer, D.; Frey, B.; Lell, M.; Manger, B.; Rech, J.; Naschberger, E., et al. Aggregated neutrophil extracellular traps limit inflammation by degrading cytokines and chemokines. Nat Med 2014, 20, 511-517, doi:10.1038/nm.3547.
36. Laridan, E.; Martinod, K.; De Meyer, S. F. Neutrophil Extracellular Traps in Arterial and Venous Thrombosis. Semin Thromb Hemost 2019, 45, 86-93, doi:10.1055/s-0038-1677040.

### VI. Working Example 2: PDO Scaffolds as a Vascular Graft for Facilitating Transmural Ingrowth of Capillaries to Generate an Endothelialized Neointimal Surface

Scaffolds made with the method of electrospinning of the present disclosure were studied. This study assessed the tensile strength, percent elongation, yield stress, yield elongation, and Young's modulus effect of said scaffolds.

### INTRODUCTION

The ideal “off-the-shelf” tissue engineering, small-diameter (SD) vascular graft hinges on designing a scaffold to act as a template that facilitates transmural ingrowth of capillaries to regenerate an endothelized neointimal surface. Towards this goal, two types of near-field electrospun (NFES) polydioxanone (PDO) architectures were explored, as SD vascular graft scaffolds. The first architecture type was a 200×200 μm and 500×500 μm grid geometry with random fiber infill, while the second architecture was aligned fibers written in a 45°/45° and 20°/70° offset from the long axis written, both on a 4 mm diameter cylindrical mandrel. These vascular graft scaffolds were evaluated for their effective pore size, mechanical properties, and platelet-material interactions compared to traditionally electrospun (TES) scaffolds and GORE-TEX® vascular grafts. It was found that effective pore size, given by 9.9 and 97 μm microsphere filtration through the scaffold wall for NFES scaffolds, was significantly more permeable compared to TES scaffolds and GORE-TEX® vascular grafts. Furthermore, ultimate tensile strength, percent elongation, suture retention, burst pressure, and Young's modulus were all tailorable compared to TES scaffold characterization. Lastly, platelet adhesion was attenuated on NFES scaffolds compared to TES scaffold, which approximates the low level of platelet adhesion measured on GORE-TEX®, with all samples showing minimal platelet activation given by P-selectin surface expression. Together, these results suggest a highly tailorable process for the creation of the next generation of small-diameter vascular grafts.

Cardiovascular disease (CVD) is a growing condition caused by narrowing blood vessels in a process termed atherosclerosis [1]. Current surgical interventions when less invasive options are no longer viable include bypass surgery with either autografts or “off-the-shelf” manufactured grafts. Autologous sources of vascular replacements are often limited because of disease or the necessity for multiple replacements per patient. Current manufactured, off-the-shelf products such as DACRON® polyethylene terephthalate (referred to herein as “DACRON®”) or GORE-TEX® expanded polytetrafluoroethylene (ePTFE) (referred to herein as “GORE-TEX®”) work adequately in adults for graft inner diameters larger than 6 mm, but smaller diameters experience high failure rates. In the short term, these grafts fail due to thrombosis, and long term they fail due to issues stemming from mechanical property mismatch [2, 3]. To address these shortcomings, the ideal solution would be an off-the-shelf, bioresorbable graft that would allow for blood to flow without thrombus formation to profuse downstream tissues while serving as a template to guide in situ regeneration of a functional artery.

Towards regeneration in animal models, the endothelial cells and smooth muscle cells migrate inwards from the anastomosis of the native vessels into ends of the graft in a process known as transanastomotic ingrowth [4, 5]. This mechanism is sufficient to restore the endothelial surface in many models, but reliance on this type of migration in humans is limited to millimeters [6, 7]. As an alternative mode, cell ingrowth can occur through transmural migration where capillaries grow through the exterior walls of the vessel or graft and sprout to source endothelial cells. Consequently, it is believed that this mode of regeneration is the primary regenerative mechanism in humans and needs to be incorporated into vascular graft design [8-10]. Therefore, the greatest design challenge for an ideal off-the-shelf vascular graft features creating scaffolds with sufficiently large pore sizes to facilitate transmural capillary ingrowth for neointimal endothelialization while maintaining robust mechanical properties with a non-thrombogenic lumen [9].

Traditional electrospinning (TES) is a popular method for creating highly porous vascular graft scaffolds to facilitate cellular ingrowth [11-13]. Nevertheless, these scaffold's fiber diameter and pore size, while tailorable, are intrinsically linked which severely limits the range of fiber diameters to those that produce large pore sizes. Even so, these pores are still restrictively under the 60-200-micron size pore for facilitating angiogenesis [14-16]. Alternatively, the relatively recent sub-technique near-field electrospinning (NFES) takes the air gap distance of TES and shortens it to a few millimeters [17]. This reduced air gap is then paired with precise relative motion between the charged polymer capillary and the grounded collector allowing for the direct writing of fibers [18]. Therefore, this technique allows for another dimension of scaffold tailorability as individual fibers can be programmed with great precision to form 3D scaffolds that are laid down layer-by-layer.

Disclosure from Working Example 1 provides a hybrid fiber architecture consisting of a highly aligned grid structure with a random fiber infill from a single set of processing parameters, FIGS. 10A-10B. The properties of this NFES scaffold's architecture allow for varying grid size to tailor pore size and mechanical properties independent of fiber diameter. Thus, in this working example, it is shown that leveraging the NFES technique to create 4 mm ID, cylindrical scaffolds with custom programmed architectures would result in non-thrombogenic small-diameter vascular grafts with tailorable pore sizes and mechanical prosperities. Towards this goal, it is demonstrated the use of a custom-built NFES apparatus built around a commercial 3D printer to create seamless small-diameter vascular graft scaffolds on a 4 mm cylindrical mandrel. Two classes of architecture were explored: the hybrid grid geometry mapped onto a cylindrical mandrel as well as an aligned fiber wind angle geometry. The grid geometries had a programmed spacing of 200 μm×200 μm (NFES 200²) and 500 μm×500 μm (NFES 500²) informed from Working Example 1, and the wind angles of 45°/45° (NFES 45°/45°) and 20°/70° (NFES 20°/70°) were chosen to explore the physiologically informed extremes of arterial ECM alignment [19]. This portfolio of architectures resulted in scaffolds with tailorable pore sizes that exceed TES scaffolds of the same fiber diameter and wall thickness as well as exceed GORE-TEX® permeability to 9.9 and 97 μm microspheres. It is believed that these pore sizes would be sufficient to facilitate cell infiltration and transmural angiogenesis. Furthermore, the NFES scaffolds resulted in tailorable mechanical properties which resulted in a better approximation of the internal mammary artery (IMA) compared to TES scaffolds. Lastly, these NFES scaffold architectures showed attenuation in the degree of static platelet adhesion with all materials showing minimal activation given by platelet P-selectin surface expression.

### Methods

Vascular Graft Scaffold Prototype Fabrication. Two consumer 3D printers (Prusa 8″ i3v Kit V-Slot Extrusion and Prusa 12″ Basic Pegasus and Makerfarm, South Jordan, Utah, USA) were modified with a custom made NFES print head comprising a remote head syringe pump (Legato 130, KD Scientific Inc, Holliston, Mass., USA) fixed on a custom-designed gantry fixation apparatus [20]. The syringe pump held a polypropylene syringe and a blunt Luer-lock needle charged by a DC voltage source (HV050REG(−, +), Information Unlimited, Amherst, N.H., USA). The NFES print head was able to translate in the X- and Z-axis, while the grounded mandrel rotated in the A-axis. The A-axis for the Prusa 8″ i3v was based on the original Y-axis stepper motor and was used for the creation of grid tubes were programmed with X- and Y-spacing of 200×200 μm and 500×500 μm. The A-axis for the Prusa 12″ Basic Pegasus was based on a stepper motor with integrated drive and encoder (STM17S-1AE, Applied Motion Products, Watsonville, Calif., USA) used for the creation of NFES scaffolds with wind angles 45°/45° and 20°/70° from the long axis. The translational path was written in G-code and sent to the 3D printer using the 3D print software Repetier (Hot-World GmbH and Co. KG, Willich, Germany), and the A-axis for the Prusa 12″ Basic Pegasus was written in the software Q Programmer (Applied Motion Products, Watsonville, Calif., USA).

Polydioxanone (DIOXOMAXX 100, Inherent viscosity 2.13 dL/g, Bezwada Biomedical, LLC, Hillsborough, N.J., USA) solutions were dissolved overnight in 1,1,1,3,3,3-hexafluoro-2-propanol (HFP, Oakwood Products, Inc., Estill, S. C., USA) at a concentration of 112 mg/mL for all NFES templates. Grid style templates were created with an air gap was set at 3 mm, an applied voltage of +1.7 kV, a polymer flow rate of 25 μL/hr through a 23-gauge, 2-inch blunt needle, and resultant velocity of 80 mm/s. Fibers were deposited on a tight-tolerance 4 mm diameter stainless steel rod (Cat No. 2961N12, McMaster-Carr, Elmhurst, Ill., USA) in a custom-built stepper motor-driven mandrel housing. Wind style templates were created with an air gap of 2.2 mm, an applied voltage of −1.4 kV, a polymer flow rate of 25 μL/hr through a 23-gauge, 2-inch blunt needle, and a resultant velocity of 80 mm/s. Fibers were deposited on a tight-tolerance 4 mm diameter stainless steel rod in a custom-built mandrel housing. TES PDO templates were fabricated with a PDO concentration of 140 mg/mL at an applied voltage of +25 kV, 17.8 cm air gap, a polymer flow 4 mL/h, through an 18 gauge, 2″ length needle. TES fibers were collected on a grounded, 4 mm stainless steel mandrel rotating at 1250 rpm, and translating 6.5 cm/s over a distance of 13 cm. After fabrication, templates were placed in a room-temperature vacuum chamber (ISOTEMP® Vacuum Oven Model 281A, Fisher Scientific, Waltham, Mass., USA) pressure to remove any remaining solvent for 10 minutes at a minimum of 70 kPa below atmospheric pressure. Scaffolds were immediately stored in a desiccant chamber until use.

A small sample was dissected from all fabricated templates for imaging followed by sputter coated in an argon atmosphere with 5.0 nm of 60:40 gold-palladium. Samples were visualized using a scanning electron microscope (Nova Nano 650 FEG, FEI Co., Hillsboro, Oreg., USA) with the field emission gun at +20 kV, a spot size of 3, and 5 mm working distance. Fiber diameters were measured from acquired images using the software Fibraquant v1.3.149 (nanoScaffold Technologies, Chapel Hill, N.C., USA). Fiber average and standard deviations were calculated from a minimum of 60 semi-automated random measurements per image calibrated from the image scale bar. All templates used had measured fiber diameters with an average between 1-2 μm, and had wall thickness between 0.1 mm-0.15 mm.

Microsphere Permeability Effective Pore Size Analysis. Fluorescent microsphere filtration was used to ascertain the effective restriction size of an object transiting the vascular graft wall [21]. Green fluorescent microspheres with 9.9 and 97 μm diameters (Cat No. G100, 35-11, Fisher Scientific, Waltham, Mass., USA) were diluted to working concentrations in deionized (DI) water per manufacture instructions. Sizes were chosen as 9.9 μm spheres were on the order of magnitude of the finest capillaries, and 97 μm spheres on the magnitude of capillaries with supporting pericytes [14-16]. Vascular grafts were cut along the long axis and were punched out into 8 mm disks (n=5) using a medical biopsy punch (Acu-Punch, 8.0 mm, Acuderm Inc., Fort Lauderdale, Fla., USA). Scaffolds were then pre-hydrated in DI water and placed inside a custom machined apparatus designed to securely hold thin membranes for filtration evaluation through a 6 mm diameter hole. A polypropylene syringe was loaded with 1.6 mL of either 500 microspheres/mL of 97 μm or 50,000 microspheres/mL of 9.9 μm spheres and was placed into an upright syringe pump (NE-300 Just Infusion™, New Era Pump Systems, Inc., Farmingdale, N.Y., USA) programmed with a flow rate of 0.25 mL/min. One milliliter of microsphere suspension was passed through the scaffolds with the remaining volume accounted for apparatus dead space. In between samples, the apparatus was flushed with 10 mL of DI water to remove any adherent microspheres.

The four cylindrical NFES geometries were evaluated along with TES templates of equivalent fiber diameters and punches from GORE-TEX® 10 mm thin wall vascular graft with rings removed (Cat No. RRT10070080L, W. L. Gore & Associates, Inc., Flagstaff, Ariz., USA). The absence of a membrane served as a positive control and 7-8 μm filter paper served as a negative control (Cat No. 09-803-6F, Fisher Scientific, Waltham, Mass., USA). The filtrate was transferred to a black with clear bottom 96 well plate and measured in duplicate using a spectrophotometer (SpectraMax i3, Molecular Devices, San Jose, Calif., USA) to scan each well area with 37 measurements from a 1 mm beamwidth using an excitation of 468 nm and an emission of 508 nm. Fluorescent values were compared to a standard curve of known microsphere counts followed by normalization to the area of flow.

Mechanical Characterization. Materials were mechanically evaluated in tension until failure. All testing was performed using a uniaxial testing frame in tension, equipped with a 25 lbf load cell (Model SM-25-294, Test Resources). All materials (n=6) were submerged in phosphate buffer saline (PBS) and placed in a 37° C. incubator for a minimum of 90 minutes to equilibrate to physiological temperature. NFES and TES scaffolds were compared to the vascular gold standard GORE-TEX® 4 mm standard wall vascular graft (Cat No. V04070L, W. L. Gore & Associates, Inc., Flagstaff, Ariz., USA). Testing methodology adhered closely to the American National Standards Institute (ANSI)/Association for the Advancement of Medical Instrumentation (AAMI) ANSI/AAMI VP20:1994 entitled ‘Cardiovascular Implants—Vascular Graft Prostheses’ [22]. Measured mechanical properties were further compared to literature values for the IMA as a physiologic reference [23-25].

Longitudinal Uniaxial Elongation. Vascular graft scaffolds were cut perpendicular to the long axis into 20 mm cylindrical segments and each end was affixed in a knurled grip vice with an initial grip separation distance of 8 mm. Grips were separated at a rate of 50 mm/min until failure and the data recorded in Newtons (N) at a sampling rate of 50 samples per second. Data were reported in megapascals (MPa) based on the initial cross-sectional area.

Circumferential Uniaxial Elongation. Vascular graft scaffolds were cut perpendicular to the long axis into 4 mm cylindrical segments and slid over two 3/64″ dowels. Dowels were secured in a custom apparatus and mounted in the knurled grip vices of the testing frame. The grips were separated at a rate of 50 mm/min until failure and the data recorded in N at a sampling rate of 50 samples per second. Data were reported in megapascals (MPa) based on the initial cross-sectional area.

Suture Retention. Vascular graft scaffolds were cut perpendicular to the long axis into 20 mm cylindrical segments and a single wall was threaded with 5-0 Surgical Steel 316L Stainless Steel monofilament (Ethicon, Somerville, N.J., USA), to ensure that suture deformation wouldn't confound in the data, 2 mm below the cut line. The sample was affixed in a knurled grip vice and the suture was secured to the opposing knurled grip vice. Grips were separated at a rate of 150 mm/min until failure and the data recorded and reported in grams-force (gf) at a sampling rate of 250 samples per second.

Burst Pressure. Vascular graft scaffolds 40 mm in length were wetted with 100% ethanol and placed over 160Q latex balloons (Qualatex, Wichita, Kans., USA). Samples were placed over barbed fittings of a custom-designed burst pressure apparatus and secured with 2-0 silk suture. The pressure was increased at a rate of 15 millimeters of mercury per second (mmHg/s) until the grafts burst and the data were measured and reported in mmHg with a pressure transducer (Cat No. 3196K93, McMaster-Carr, Elmhurst, Ill., USA) at a sampling rate of 100 samples per second.

### Human Platelet Adhesion and Activation Response

Platelet Isolation and Seeding. Blood was donated from Tennessee Blood Services from healthy donors. Since purchased or donated samples are not traceable back to the donor, it does not qualify as human subjects research as determined by the University of Memphis Institutional Review Board on Nov. 22, 2016. Human peripheral blood platelets were isolated as platelet-rich plasma (PRP) from whole blood obtained via venipuncture through density centrifugation at 150 g for 17 minutes at room temperature from citrated whole blood. The bottom third of the plasma fraction was kept and the remaining plasma fraction was further centrifuged at 700 g for 17 minutes to create platelet-poor plasma (PPP).

The cylindrical vascular graft scaffolds were cut along their long axis and 8 mm discs were punched out using a medical biopsy punch. Samples were UV sterilized (EN-280L 1090 μW/cm² at 15 cm, SPECTROLINE®, Westbury, N.Y., USA) for 10 minutes on each side at a distance of 10 cm from the source and placed in a 96-well plate before seeding. Scaffold punches were hydrated with 50 μL of platelet-poor plasma and subsequently were seeded with 100 μL of 1×10⁷ platelets/mL (1,000,000 platelets/well). As the reference architecture, TES scaffolds with 500 nM Phorbol 12-myristate 13-acetate (PMA) served as the positive control, as PMA has been shown to activate platelets with a sustained surface expression of P-selectin (CD62P) [26, 27]. Also, TES scaffolds identical concentration dimethyl sulfoxide (DMSO) served as vehicle control. Platelets were in contact with scaffolds for 15 and 30 minutes under standard culture conditions of 37° C. and 5% CO₂.

Scaffold Staining, Microscopy, and Analysis. After incubation, scaffolds were washed with PBS for 60 seconds with gentle agitation 5 times to remove any non-adhered platelets. The remaining scaffold-bound platelets were fixed in their wells with 10% buffered formalin for 30 minutes. Samples were washed in 100 mM glycine in PBS for 5 minutes 3 times to quench free aldehyde groups followed by incubation with 5% bovine serum albumin (BSA) for 1 hour at room temperature with gentle agitation to block non-specific binding. After blocking, samples were washed with PBS and covered with a 1:100 dilution of anti-human CD62P (Cat No. 304902, Biolegend, San Diego, Calif., USA) in 1% BSA at 4° C. overnight with gentle agitation. Following, samples were washed with PBS 3 times for 5 minutes and were covered with 1:200 diluted fluorophore-conjugated secondary antibody (Cat No. A11030, Invitrogen, Carlsbad, Calif., USA) in PBS with 1% BSA. Samples were incubated for 1 hour at room temperature with gentle agitation. After incubation samples were washed with PBS 3 times for 5 minutes and stained with 4′,6-diamidino-2-phenylindole (DAPI) (NucBlue™ Fixed Cell Stain ReadyProbes™, Molecular Probes, Eugene, Oreg., USA) per manufacture concentration for 5 minutes. Lastly, cells were permeabilized with 0.1% Triton X-100 for 10 minutes at room temperature and stained with Actin green per manufacture concentration for 30 min (ActinGreen™ 488 ReadyProbes™, Invitrogen, Carlsbad, Calif., USA). Scaffolds were stored in 96 well plates immersed in PBS at 4° C. and were imaged within 72 h.

Scaffolds were placed on a glass slide mounted with PBS and covered with a glass cover slide. Fluorescently stained cells on scaffolds were imaged with an Olympus BX 63 fluorescent microscope (Olympus Corporation, Center Valley, Pa., USA) with an Olympus DP80 CCD camera using a 20× objective. Images were acquired in cell Sens Dimensions Version 2.3 and saved as lossless VSI and tiff file formats. Exposure was set based on being below the saturation limit of the CCD camera for the PMA positive control. Scaffolds were imaged in 3 random locations with a 25 μm Z-stack of images. Step size was determined by the Nyquist frequency of the limiting wavelength of the DAPI filter. Olympus's cellSens TruSight deconvolution software was used to further process the Z-stack images using the Constrained Iterative Filter for 8 iterations followed by forming a 2D extended focal image (EFI) from the deconvolved Z-stacks. Images were analyzed using the program CellProfiler™ v3.1.8 (Broad Institute) [28, 29]. The degree of platelet adherence to materials was given by material coverage of actin cytoskeleton staining in the green channel, while platelet activation was measured by material coverage of cell surface expression of P-selectin in the red channel.

Statistics. All statistical analysis was performed in Prism 8 v8.2.1 (GraphPad Software Inc., San Diego, Calif., USA). Differences were tested between groups using ANOVA with Holm-Sidak's multiple comparisons at a significance of p<0.05. Grid sizes and wind angles were compared to their theoretical programmed value using a one-sample t-test at a significance of p<0.05. Data were reported as mean±standard deviation.

### Results

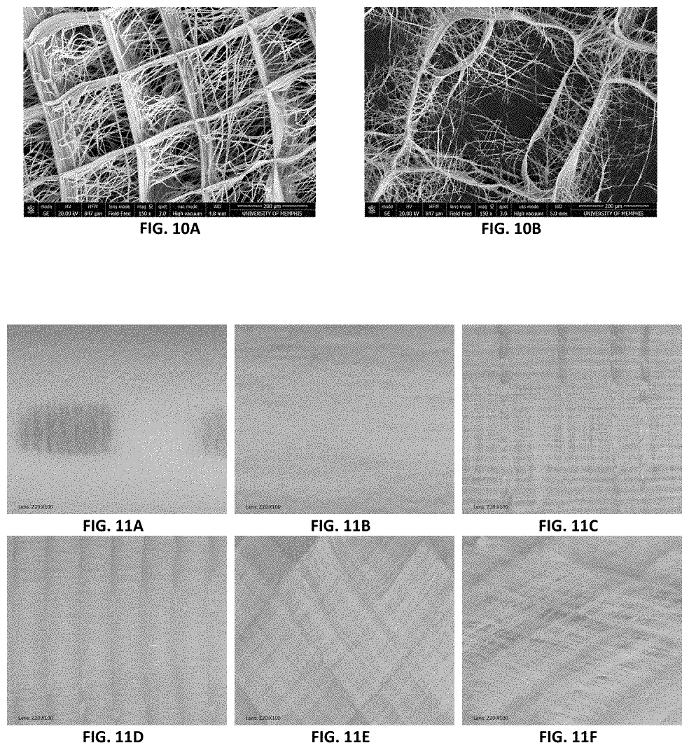

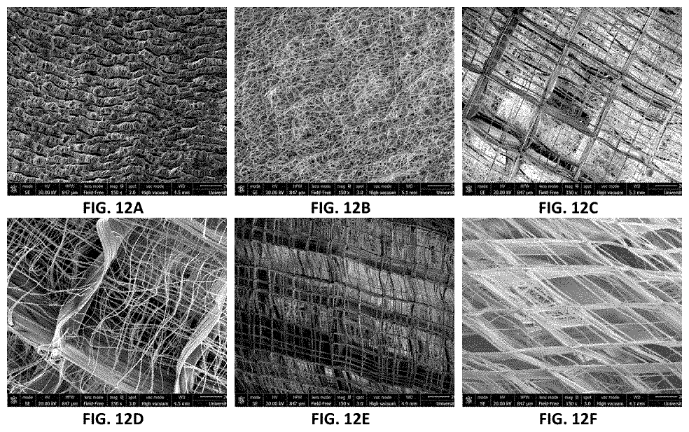

Vascular Graft Scaffold Fabrication. Qualitative evaluation of 4 mm ID vascular graft scaffolds by digital microscopy showed GORE-TEX® expanded polytetrafluoroethylene (ePTFE), randomly distributed TES fibers, and NFES fibrous scaffolds with well-defined grid and wind architectures, FIGS. 11A-11F. Qualitative evaluation of tubular samples by SEM revealed GORE-TEX® ePTFE with manufacture listed 25 μm nodes, TES scaffolds with random fibers free of defects, and NFES scaffolds showed either regular grids with random fiber infill or aligned fibers at a particular angle free of defects, FIGS. 12A-12F. Quantitatively, SEM images showed fiber diameter averages between all TES and NFES scaffolds that fell within the cutoff limit of 1-Geometrically there was no significant difference (p>0.05) between the measured NFES 200² and 500² μm grids compared to the theoretical programmed geometry of 200×200 μm and 500×500 respectively as well as NFES 45°/45° and 20°/70° scaffolds compared to the theoretical programmed angles. These results suggest that a diverse portfolio of geometries can be created as a function of programming while maintaining a constant fiber diameter.

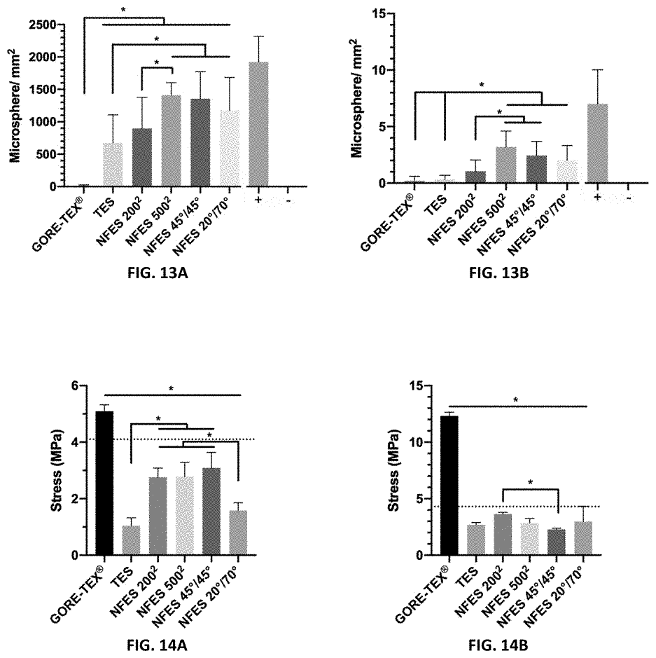

Vascular graft wall permeability for 9.9 μm microspheres showed that all TES and NFES scaffolds were significantly more permeable compared to GORE-TEX® which was virtually impermeable to the 9.9 μm microspheres, FIG. 13A. All NFES scaffolds except for NFES 200² were significantly twice as permeable as TES scaffolds with a permeability of 675±432 microspheres/mm². Within NFES scaffolds only NFES 200² and 500² were significantly different as NFES 500² was 1.5-fold more permeable with 1409±194 microspheres/mm². For the 97 μm microspheres, NFES 500², 45°/45°, and 20°/70° scaffolds were significantly more permeable than GORE-TEX® and TES which were both effectively impermeable to the microspheres, FIG. 13B. Within NFES scaffolds, 500² and 45°/45°, were significantly more permeable to the 97 μm microspheres than NFES 200² scaffolds which had a permeability of 1.1±0.9 microspheres/mm² by 2 to 3-fold. It is important to note that GORE-TEX® thin wall vascular graft's thickness of 0.45 mm compared to all NFES and TES scaffolds wall thickness of 0.1-0.15 mm makes direct comparisons not possible. Nevertheless, GORE-TEX® thin wall samples as manufactured are not permeable to both the 9.9 and 97 μm microspheres. Taken together these data suggest that NFES scaffolds are significantly more permeable to capillary-sized objects than TES scaffolds of the same fiber diameter. Furthermore, this permeability can be tailored as a function of scaffold programming independent of fiber diameter.

Mechanical Characterization of Vascular Grafts. NFES vascular graft scaffolds were mechanically evaluated and compared to TES scaffolds of the same fiber diameter range as well as commercially available matching ID GORE-TEX® vascular grafts. Ultimate tensile strength (UTS) in the circumferential axis showed that GORE-TEX® was significantly stronger by 5-fold compared to TES and 1.5-3-fold compared to NFES scaffolds, FIG. 14A. All NFES scaffolds had a significantly greater UTS compared to TES, which had a value of 1.0±0.3 MPa, by 2.5-3-fold except for NFES 20°/70°, which was not statistically different. Furthermore, within the NFES scaffolds, NFES 20°/70° with a value of 1.6±0.3 MPa had a significantly lower UTS by half compared to 3.1±0.6 MPa of NFES 45°/45°. When compared to IMA reference value of 4.1 MPa, NFES 200², 500², and 45°/45° had lower measured values of 1.3-1.5-fold decrease than that of the IMA, while TES and NFES 20°/70° scaffolds had measured values 2.6-3.9-fold less than that of the IMA. GORE-TEX® vascular grafts were the only group that had a UTS of 1.2-fold greater than the IMA reference value. In the longitudinal axis, GORE-TEX® had a significantly greater UTS of 3.3 to 5.3-fold compared to TES and NFES scaffolds, FIG. 14B. Furthermore, GORE-TEX® UTS was 3-fold greater than the IMA reference value of 4.3 MPa. There was no significant difference between TES and all NFES scaffolds in the longitudinal axis, and within NFES scaffolds, NFES 200² with a UTS of 3.7±0.1 MPa was significantly greater by 1.6-fold compared to NFES 45°/45°. All TES and NFES scaffolds resulted in a 1.2-1.9-fold lower longitudinal UTS compared to the IMA.

The data for percent elongation at failure in the circumferential axis showed that all TES and NFES scaffolds were significantly different from GORE-TEX®, FIG. 15A. Specifically, TES with a value of 18.8±11.8% showed a significantly reduced elongation at failure of 3.8-fold compared to GORE-TEX®, while NFES scaffolds were significantly more ductile than GORE-TEX®by 2.5 to 3.5-fold. All NFES scaffolds had a significantly greater elongation at failure of 10-13-fold over TES scaffolds. Within NFES, the wind angle 45°/45° and 20°/70° scaffolds were significantly greater than grid 200² and 500² scaffolds. Furthermore, NFES 20°/70° with a measured 257.9±41.0% had a significantly greater elongation compared to NFES 45°/45°. The NFES 200² and 500² scaffolds most closely approximated the IMA physiologic elongation value being 1.4-fold greater than the reference 134%. The wind NFES 45°/45° and 20°/70° scaffolds were 1.6-fold and 1.8-fold more ductile compared to the IMA reference value, respectively, while GORE-TEX® and TES scaffolds were 1.9-fold and 7.1-fold less ductile compared to the IMA reference value, respectively.

In the longitudinal axis, GORE-TEX® had a significantly lower elongation at failure compared to NFES 200², 500², and 45°/45° scaffolds by 1.7-2.4-fold, but there was no difference compared to NFES 20°/70°, FIG. 15B. Significant differences were found between NFES 200², 45°/45°, 20°/70° scaffolds compared to TES scaffolds which had a value of 122±20%. Specifically, the data showed that NFES 200² with a value of 231±14% and 45°/45° with a value of 217±34% had a 2-fold increase in the elongation at failure compared to TES scaffolds while NFES 20°/70° scaffolds had a 2-fold reduction in elongation compared to TES scaffolds. Within NFES scaffolds, NFES 20°/70° scaffolds were significantly less ductile with a 3-4.3-fold reduction in percent elongation compared to all other NFES scaffolds. Furthermore, within NFES scaffolds, NFES 45°/45° was significantly greater than NFES 500² by 1.3-fold. Compared to the IMA, NFES 20°/70° most closely approximated the physiological value while all other NFES groups exceeded the reference percent elongation value of 59%.

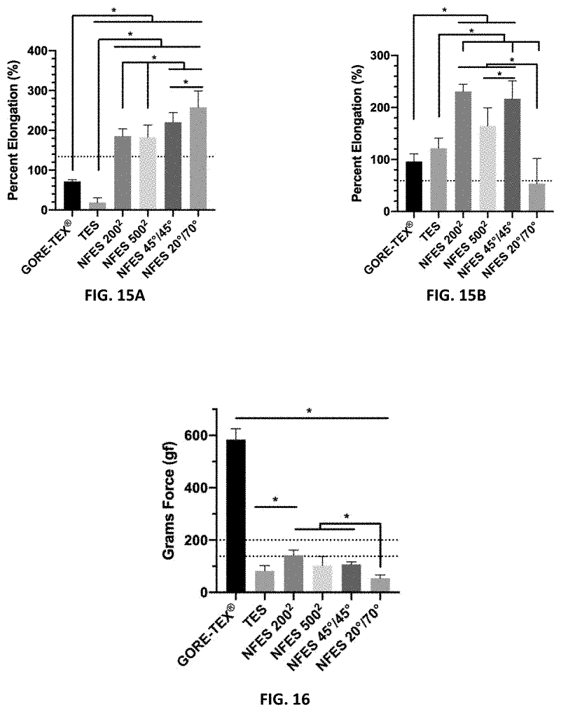

For suture retention, the data showed that GORE-TEX® had the greatest strength of 584±42 gf and was significantly greater than all other NFES and TES scaffolds, FIG. 16. Nevertheless, as the data are not normalized to thickness, direct comparisons cannot be made. Compared to TES scaffolds, only NFES 200² scaffolds with a strength of 142±20 gf were significantly greater at 1.7-fold the measured force for TES scaffolds. Within NFES scaffolds, NFES 20°/70° had a significantly lower suture retention force of 54±13 gf which was 1.9-2.6-fold less than the other NFES scaffolds. Compared to the IMA, only NFES 200² was within the reported values range of 138-200 gf. All other NFES and TES scaffolds were 1.3-2.6-fold less than the IMA lower value.

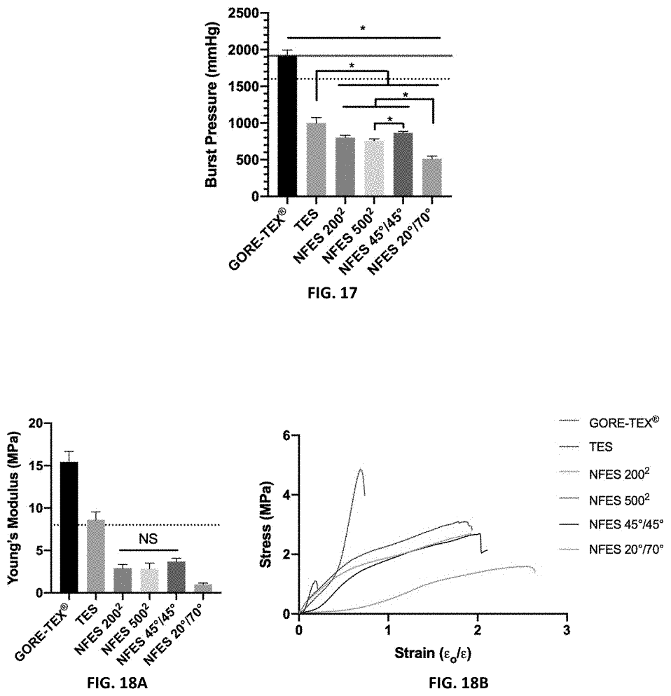

The burst pressure for GORE-TEX® vascular grafts was never reached as water began to leak around the barbed connectors that were secured by suture. Nevertheless, GORE-TEX® had a significantly greater measured pressure than all NFES and TES scaffolds by 2 to 4-fold, FIG. 17. TES scaffolds with a value of 1002±73 mmHg had a significantly greater burst pressure of 1.25-2-fold the burst pressure of NFES scaffolds. Within NFES scaffolds, NFES 20°/70° with a value of 511±38 mmHg had a significantly lower burst pressure of 1.5-1.7-fold compared to all other NFES scaffold architectures. Furthermore, NFES 45°/45° with a value of 866±21 mmHg was significantly greater than NFES 500² by 1.1-fold. Compared to the IMA with a value of 1600 mmHg, only GORE-TEX® had a burst pressure greater than the reference value. TES scaffold grafts had a 1.6-fold lower value while NFES scaffolds had between a 1.8-3.1-fold lower burst pressure value.

The data for Young's modulus in the circumferential axis showed GORE-TEX® with a significantly greater modulus by 2-fold compared to TES scaffolds and 4-15-fold greater than NFES scaffolds, FIG. 18A. The TES scaffold with a modulus of 8.6±0.9 MPa was also significantly greater than all NFES scaffolds by 2.3-8.6-fold. Within NFES scaffolds, there were no differences detected among 200², 500², and 45°/45 scaffolds, but these scaffolds were all significantly stiffer than NFES 20°/70° with a modulus of 1.0±0.2 MPa by 3-fold. Compared to the IMA's modulus, TES ideally approximated the reference modulus of 8 MPa, while all NFES scaffolds were 2-8-fold less than the native modulus. GORE-TEX® vascular grafts resulted in a 2-fold greater modulus compared to the IMA reference. Qualitatively, the representative stress-strain curves showed GORE-TEX®, NFES 45°/45°, and NFES 20°/70° groups with J-shaped stress-strain graphs typical of many biological materials as given by their toe, heel, linear, and rupture regions, FIG. 18B [30, 31]. In contrast, TES demonstrated the behavior of a rigid plastic, and NFES 200², as well as NFES 200², demonstrated the behavior of a more flexible plastic.

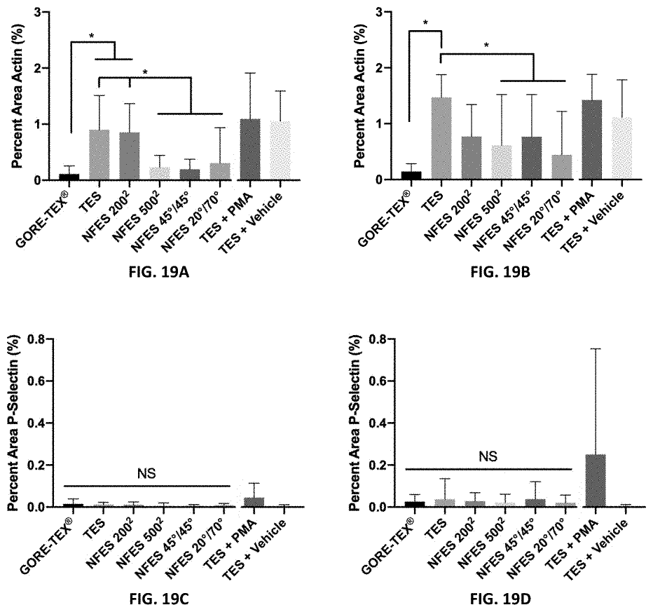

Human Platelet Adhesion and Activation to Vascular Grafts. The static adhesion and activation of platelets interacting with vascular graft scaffolds were evaluated at 15 and 30 minutes, FIGS. 19A-19D. GORE-TEX® had a significant 9-fold lower percent of adhered platelets at 15 minutes compared to TES with 0.9±0.6% and NFES 200² 0.9±0.5% area coverage. All other comparisons of NFES scaffolds to GORE-TEX® had no statistical difference. Comparisons to TES scaffolds showed no significant difference compared to NFES 200². All other NFES scaffold architectures showed a significantly lower degree of platelet adhesion compared to TES by 3-4.6-fold. Within NFES scaffolds, NFES 200² showed a significantly greater degree of platelet surface coverage compared to all other NFES scaffolds by 3-4.6-fold. Platelet activation given by coverage of platelet surface-expressed P-selectin showed no detectable differences in the degree of coverage among all groups. Percent area coverage values ranged from 0.005-0.015% indicating a low degree of activation from static material contact.

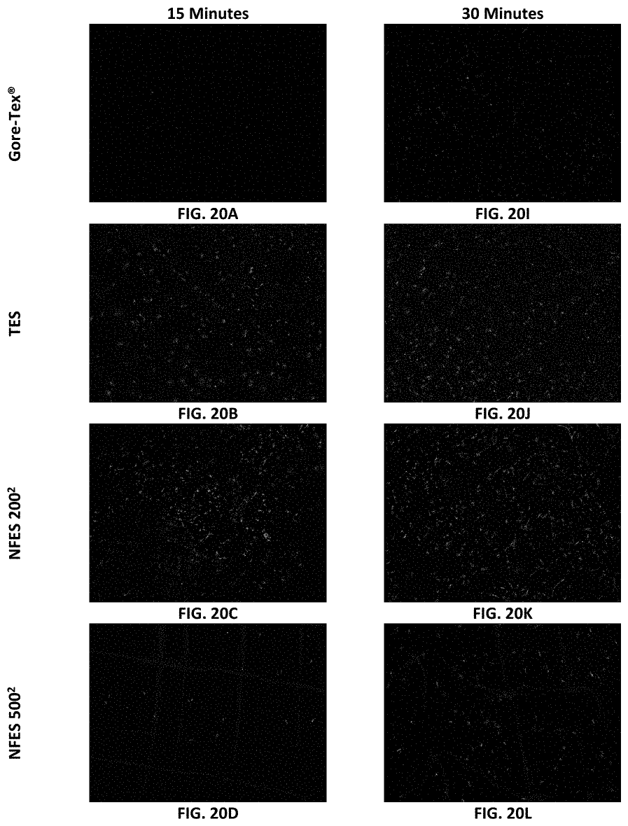

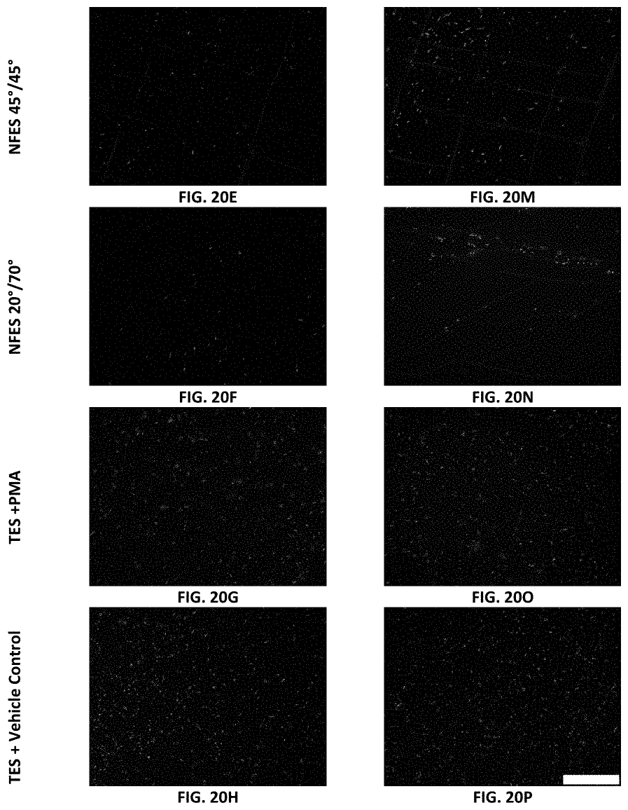

At 30 minutes, GORE-TEX® had a significant 11-fold decrease in the area of adhered platelets compared to the 1.5±0.4% platelet coverage of TES scaffolds. There were no statistical differences detected between GORE-TEX® and all NFES scaffolds. The data for TES scaffolds showed no difference compared to NFES 200², and TES was significantly greater than all other NFES scaffolds by 3-5-fold. Within NFES scaffolds, there were no statistical differences detected in platelet percent area coverage. Similar to the 15-minute time point, coverage of surface-expressed P-selectin showed no detectable differences in the degree of coverage among all groups. Percent area coverage values ranged from 0.02-0.03% indicating a low degree of activation from static material contact. Trends from the quantitative data can be observed qualitatively in the representative images, FIGS. 20A-20Q. The GORE-TEX® vascular grafts demonstrated the least degree of platelet adherence at both time points. The smaller pore size scaffold architectures TES and NFES 200² had a greater degree of adhered platelets at both time points compared to GORE-TEX® as well as the large pore size NFES architectures. No evaluated scaffold grafts had a surface area coverage of activated platelets above 0.03% suggesting a favorable material interaction under static conditions.

### DISCUSSION

The current gold standard materials for vascular grafts are polyethylene terephthalate (PET, brand name DACRON®) and expanded polytetrafluoroethylene (ePTFE, brand name GORE-TEX®). These materials work adequately for grafts with inner diameters larger than 6 mm, but smaller diameters experience high failure rates. Acutely, these grafts fail via thrombosis while chronically they fail due to issues stemming from mechanical property mismatch [2, 32]. Thus, there is a further need to develop biomaterials with optimal tissue-material interactions that can succeed in these small diameter graft applications.

The ideal non-thrombogenic surface is the endogenous endothelium, therefore restoring this surface is paramount to long-term success [33, 34]. It has been rigorously demonstrated that re-endothelialization can occur through the ingrowth of capillaries into a vessel wall in a process termed transmural endothelialization, and it is anticipated to be the primary mechanism in humans [5, 9, 10, 35, 36]. This is opposed to other mechanisms of re-endothelialization such as the ingrowth of endothelial cells from the adjoining vessel, termed transanastomotic endothelization, a process prominently seen in animal models [4]. Specifically, transmural endothelialization was demonstrated by Pennel et al. using an isolated loop graft model. In these two material grafts, a high porosity polyurethane central region contained pore sizes of 150 μm and 75 μm interconnects, this was flanked by GORE-TEX® with 30 μm pores [9, 37]. The high porosity isolated region and anastomotic edge were the first to endothelize before the remaining GORE-TEX® regions in a Wistar rat model. Histology revealed capillary ingrowth into the central region as the source of the endothelial cells. Thus, indicating the importance of developing highly porous scaffolds to facilitate this ingrowth of capillaries.

The creation of highly porous scaffolds is a central tenet of tissue engineering as these attributes affect cell infiltration and nutrient/oxygen exchange. Electrospun scaffolds, while highly porous, have seen limited success as tailorable pore sizes are proportional to the relationship between fiber diameters and packing density [38, 39]. Towards the creation of large pores, Pham et al. showed with mercury porosimetry data that 10 μm diameter fibers were required to achieve a 45 μm pore size average. Similarly, a compilation of mercury porosimetry modeling and experimental data by Szentivanyi et al. showed that electrospun scaffolds composed of 40 μm diameter fibers would be required to achieve pore sizes of 100 μm [40]. Fibers this large are impractical to make and well beyond a biologically relevant diameter. Thus NFES's process of directly writing fibers de-couples the relationship between fiber size and pore size [41]. Pore size becomes a function of the preprogrammed fiber path and fiber size remains a function of the processing parameters. To date, the vast majority of published fiber writing geometries feature perfectly stacked grids or triangles [42, 43]. While these architectures are elegant in form, they are unfavorable for a blood-tight vascular graft and cellular ingrowth.

Vascular extracellular matrix (ECM) architecture has been shown by Timmins et al. that intima-media elastin and collagen adopt a longitudinal orientation and then subsequently transition to a circumferential orientation at the interface with endothelial cells [44]. Furthermore, a review by Rhodin et al. compiled the aggregate data which suggested that vascular smooth muscle cells (vSMC) in the intima-media adopt a 20-40° angle from the long axis [19]. Therefore, it was believed that our hybrid grid geometry would approximate the longitudinal/circumferential architecture, while aligned fiber wind angles of 20°/70° through 45°/45° would approximate the range of vSMC orientations to together provide a basis of architectures for the creation of non-thrombogenic vascular graft scaffolds with highly tailorable pore sizes and mechanical properties. Towards this belief, it was demonstrated that NFES 500², 20°/70°, and 45°/45° scaffolds were permeable to the 97 μm microspheres while the smaller pore size NFES 200² scaffolds were less permeable. The TES scaffolds, as well as GORE-TEX® vascular grafts, were impermeable to the 97 μm microsphere which is consistent with Pennel et al. showing no transmural capillary ingrowth into GORE-TEX® while the more porous region with 75 μm interconnects did result in ingrowth. Thus, it is believed that the disclosed NFES 500², 20°/70°, and 45°/45° scaffolds and to a lesser degree NFES 200² scaffolds would be sufficiently porous to facilitate transmural angiogenesis.

Subsequently, alternate failure modes of vascular grafts stem from a mechanical mismatch of the graft and anastomoses vessel. While a bioresorbable graft only temporarily has to serve as a conduit for blood, mechanical properties need to be extensively tuned to provide the correct mechanical signals for cells and not prematurely fail before a neovessel can be formed [45]. In this work, it was demonstrated that a diverse portfolio of scaffold architectures resulted in the ability to tailor program mechanical properties. Compared to TES scaffolds, NFES scaffolds resulted in a greater UTS, percent elongation at failure, and suture retention with only a 25% reduction in burst pressure. NFES scaffolds compared to the IMA, underperformed in UTS and burst pressure. Alternatively, GORE-TEX® vascular grafts had extensively greater UTS, suture retention, modulus, and burst pressure compared to NFES scaffolds as well as compared to the IMA physiologic reference.

Lastly, as transmural endothelization is occurring, a tissue engineering vascular graft scaffold must have limited thrombogenicity to bridge the transition. Pore and fiber size have also been associated with influencing the thrombogenic innate immune response as Milleret et al. demonstrated excessive thrombin formation and platelet adhesion associated with large fiber diameters TES fibers greater than 2 [46]. Our data show that higher pore size NFES 500², 45°/45°, and 20°/70° had minimal platelet adhesion surface coverage similar to GORE-TEX® at both 15 and 30 minutes under static conditions. Further still, all NFES and TES scaffolds had less than 0.04% surface area coverage P-selectin surface-expressed plated with no difference compared to GORE-TEX®. This absence of spontaneously activated platelets on contact with these materials is a favorable initial evaluation of blood-material interactions but needs to be followed by evaluation for thrombogenicity under dynamic flow conditions.

### CONCLUSION

The ideal “off the shelf,” bioresorbable, tissue engineering solution for a small diameter vascular graft hinges on designing a scaffold to serve as a template that directs the restoration of the endothelial surface with the appropriate attenuation of mechanical properties as the scaffold is replaced with a native functional blood vessel. Towards this goal, our NFES vascular graft scaffolds demonstrated the creation of expansive pores that are anticipated to facilitate transmural endothelialization through bioinstructive design with tailorable mechanical properties that approximate native values. Further studies are expected to further approximate mechanical properties through exploring scaffold wall thickness as well as demonstrate transmural capillary ingrowth in an in vitro followed by in vivo model. It is believed that the ability to custom-program and tailor NFES scaffolds are the future for in situ regeneration and the next generation of small-diameter vascular grafts.

### REFERENCES

1. Heart disease. 12-19-19]; Available from: https://www.mayoclinic.org/diseases-conditions/heart-disease/symptoms-causes/syc-20353118.
2. Bush Jr, H., Mechanisms of graft failure. RESEARCH INITIATIVES IN VASCULAR SURGERY, 1989. 9ths (2): p. 392-394.
3. Handa, R. and S. Sharma, Vascular Graft Failure of Leg Arterial Bypasses—A Review. Journal of Hypertension and Cardiology 2014. 1(3).
4. Koobatian, M. T., et al., Successful endothelialization and remodeling of a cell-free small-diameter arterial graft in a large animal model. Biomaterials, 2016. 76: p. 344-58.
5. Sanchez, P. F., E. M. Brey, and J. C. Briceno, Endothelialization mechanisms in vascular grafts. J Tissue Eng Regen Med, 2018. 12(11): p. 2164-2178.
6. Zilla, P., D. Bezuidenhout, and P. Human, Prosthetic vascular grafts: wrong models, wrong questions and no healing. Biomaterials, 2007. 28(34): p. 5009-27.
7. Davids, L., T. Dower, and P. Zilla, The lack of healing in conventional vascular grafts. Tissue engineering of vascular prosthetic grafts, 1999: p. 3-44.
8. Row, S., et al., Arterial grafts exhibiting unprecedented cellular infiltration and remodeling in vivo: the role of cells in the vascular wall. Biomaterials, 2015. 50: p. 115-126.
9. Pennel, T., P. Zilla, and D. Bezuidenhout, Differentiating transmural from transanastomotic prosthetic graft endothelialization through an isolation loop-graft model. J Vasc Surg, 2013. 58(4): p. 1053-61.
10. Pennel, T., et al., Transmural capillary ingrowth is essential for confluent vascular graft healing. Acta Biomater, 2018. 65: p. 237-247.
11. Doshi, J. and D. H. Reneker, Electrospinning Process and Apllications of Electrospun Fibers. Journal of Electrostatics, 1995. 35: p. 151-160.
12. Ding, J., et al., Electrospun polymer biomaterials. Progress in Polymer Science, 2019. 90: p. 1-34.
13. Sell, S. A., et al., The Use of Natural Polymers in Tissue Engineering: A Focus on Electrospun Extracellular Matrix Analogues. Polymers, 2010. 2(4): p. 522-553.
14. Oliviero, O., M. Ventre, and P. A. Netti, Functional porous hydrogels to study angiogenesis under the effect of controlled release of vascular endothelial growth factor. Acta Biomater, 2012. 8(9): p. 3294-301.
15. Xiao, X., et al., The promotion of angiogenesis induced by three-dimensional porous beta-tricalcium phosphate scaffold with different interconnection sizes via activation of PI3K/Akt pathways. Sci Rep, 2015. 5: p. 9409.
16. Walthers, C. M., et al., The effect of scaffold macroporosity on angiogenesis and cell survival in tissue-engineered smooth muscle. Biomaterials, 2014. 35(19): p. 5129-37.
17. Kameoka, J., et al., A scanning tip electrospinning source for deposition of oriented nanofibres. Nanotechnology, 2003. 14(10): p. 1124-1129.
18. Sun, D., et al., Near-Field Electrospinning. Nano Lett, 2006. 6(4): p. 839-842.
19. Rhodin, J., Architecture of the vessel wall, in Handbook of Physiology, S. Geiger, et al., Editors. 1980, American Physiological Society: Bethesda, Md.
20. King, W. E., III, et al., Characterization of Polydioxanone in Near-Field Electrospinning. Polymers (Basel), 2019. 12(1).
21. Duke, S., Evaluating pore sizes of biological membranes with fluorescent microspheres. Particulate Science and Technology, 1989. 7(3).
22. Standard, A. N., Cardiovascular implants□Vascular graft prostheses. 1994.
23. Stekelenburg, M., et al., Dynamic straining combined with fibrin gel cell seeding improves strength of tissue-engineered small-diameter vascular grafts. Tissue Eng Part A, 2009. 15(5): p. 1081-9.
24. L'Heureux, N., et al., Human Tissue Engineered Blood Vessel For Adult Arterial Revascularization. Nature medicine, 2006. 12(3): p. 361.
25. Konig, G., et al., Mechanical properties of completely autologous human tissue engineered blood vessels compared to human saphenous vein and mammary artery. Biomaterials, 2009. 30(8): p. 1542-1550.
26. Whiss, P. A., R. G. Andersson, and U. Srinivas, Kinetics of platelet P-selectin mobilization: concurrent surface expression and release induced by thrombin or PMA, and inhibition by the NO donor SNAP. Cell Adhes Commun, 1998. 6(4): p. 289-300.
27. Massaguer, A., et al., Characterization of platelet and soluble porcine P-selectin (CD62P). Vet Immunol Immunopathol, 2003. 96(3-4): p. 169-81.
28. Fattahi, P., J. T. Dover, and J. L. Brown, 3D Near-Field Electrospinning of Biomaterial Microfibers with Potential for Blended Microfiber-Cell-Loaded Gel Composite Structures. Adv Healthc Mater, 2017. 6(19).
29. McQuin, C., et al., CellProfiler 3.0: Next-generation image processing for biology. PLoS Biol, 2018. 16(7): p. e2005970.
30. Zhalmuratova, D., et al., Mimicking “J-Shaped” and Anisotropic Stress-Strain Behavior of Human and Porcine Aorta by Fabric Reinforced Elastomer Composites. ACS Appl Mater Interfaces, 2019. 11(36): p. 33323-33335.
31. Ma, Y., et al., Design and application of ‘J-shaped’ stress-strain behavior in stretchable electronics: a review. Lab Chip, 2017. 17(10): p. 1689-1704.
32. Scharn, D. M., et al., Biological mechanisms influencing prosthetic bypass graft patency: possible targets for modern graft design. Eur J Vasc Endovasc Surg, 2012. 43(1): p. 66-72.
33. Boulanger, C. M., Endothelium. Arterioscler Thromb Vasc Biol, 2016. 36(4): p. e26-31.
34. Gimbrone, M. A., Jr. and G. Garcia-Cardena, Vascular endothelium, hemodynamics, and the pathobiology of atherosclerosis. Cardiovasc Pathol, 2013. 22(1): p. 9-15.
35. Matsuda, T. and Y. Nakayama, Surface microarchitectural design in biomedical applications: in vitro transmural endothelialization on microporous segmented polyurethane films fabricated using an excimer laser. J Biomed Mater Res, 1996. 31(2): p. 235-42.
36. Desgranges, P., et al., Transmural endothelialization of vascular prostheses is regulated in vitro by Fibroblast Growth Factor 2 and heparan-like molecule. Int J Artif Organs, 1997. 20(10): p. 589-98.
37. Schmidt, S., et al., Small-diameter vascular prostheses: Two designs of PTFE and endothelial cell—seeded and nonseeded Dacron. Journal of Vascular Surgery, 1985. 2(2).
38. Thompson, C. J., et al., Effect of parameters on nanofiber diameter determined from electrospinning model. Polymer, 2007. 48: p. 6913-6922.
39. Deitzel, J. M., et al., The effect of processing variables on the morphology of electrospun nanofibers and textiles. Polymer, 2001. 42: p. 261-272.
40. Szentivanyi, A., et al., Electrospun cellular microenvironments: Understanding controlled release and scaffold structure. Adv Drug Deliv Rev, 2011. 63(4-5): p. 209-20.
41. Sun, D., C. Chang, and L. Lin, Near-Field Electrospinning. Nano Lett, 2006. 6(4): p. 839-842.
42. Tylek, T., et al., Precisely defined fiber scaffolds with 40 mum porosity induce elongation driven M2-like polarization of human macrophages. Biofabrication, 2020. 12(2): p. 025007.
43. Zhang, Z., et al., 3D anisotropic photocatalytic architectures as bioactive nerve guidance conduits for peripheral neural regeneration. Biomaterials, 2020. 253: p. 120108.
44. Timmins, L. H., et al., Structural inhomogeneity and fiber orientation in the inner arterial media. Am J Physiol Heart Circ Physiol, 2010. 298(5): p. H1537-45.
45. Simons, M., E. Gordon, and L. Claesson-Welsh, Mechanisms and regulation of endothelial VEGF receptor signalling. Nat Rev Mol Cell Biol, 2016. 17(10): p. 611-25.
46. Milleret, V., et al., Influence of the fiber diameter and surface roughness of electrospun vascular grafts on blood activation. Acta Biomaterialia, 2012. 8(12): p. 4349-4356.

It will be appreciated that the scaffolds, and methods of making the scaffolds, described in detail above are merely exemplary. A variety of similar scaffolds will function in the same or similar manner. Indeed, the foregoing description illustrates and describes the processes, manufactures, compositions of matter, and other teachings of the present disclosure. Additionally, the disclosure shows and describes only certain embodiments of the processes, manufactures, compositions of matter, and other teachings disclosed, but as mentioned above, it is to be understood that the teachings of the present disclosure are capable of use in various other combinations, modifications, and environments and are capable of changes or modifications within the scope of the teachings as expressed herein, commensurate with the skill and/or knowledge of a person having ordinary skill in the relevant art. The embodiments described hereinabove are further intended to explain certain best modes known of practicing the processes, manufactures, compositions of matter, and other teachings of the present disclosure and to enable others skilled in the art to utilize the teachings of the present disclosure in such, or other, embodiments and with the various modifications required by the particular applications or uses. Accordingly, the processes, manufactures, compositions of matter, and other teachings of the present disclosure are not intended to limit the exact embodiments and examples disclosed herein. Any section headings herein are provided only for consistency with the suggestions of 37 C.F.R. § 1.77, or otherwise to provide organizational queues. These headings shall not limit or characterize the invention(s) set forth herein.

What is claimed is:

1. A method of producing a hybrid fibrous scaffold, the method comprising:
dissolving a polymer in a solution to create a polymer-containing solution;
electrically charging the polymer-containing solution; and
writing the polymer-containing solution on a counter electrode or a ground in a grid pattern to form semi-stable fibers comprised of the polymer, the semi-stable fibers comprising a plurality of bent fibers and a plurality of straight fibers and forming the hybrid fibrous scaffold.

2. The method of claim 1, wherein the writing is performed by an additive manufacturing system, and wherein the writing is performed based on programmed fiber placement.

3. The method claim 1, wherein the writing is performed in layers to form a stable layer and an unstable layer.

4. The method of claim 3, wherein the stable layer, the semi-stable layer, and the stable layer are written sequentially two or more times.

5. The method of any one of claim 1, wherein the polymer-containing solution is written in 10 layers to 10,000 layers to form the hybrid fibrous scaffold, the hybrid fibrous scaffold having a number of layers equal to the number of layers in which the solution is written.

6. The method of claim 1, wherein the polymer-containing solution is written on a counter electrode or ground of having a flat, concave, convex, or irregular surface geometry in a predetermined writing path comprising one or more of: a grid size, a scaffold size, a layer count, an air gap, an electric field strength, and a geometry.

7. The method of claim 6, wherein:
the grid size is from 50 μm×50 μm to 10,000 μm to 10,000 μm;
the scaffold size is from 20 mm×5 mm to 400 to 100 mm;
the geometry comprises a stacking grid geometry;
the air gap is from 1 mm to 10 mm;
the electric field strength is from 0.1 kV/mm to 2.0 kV/mm; or
any combination of the foregoing.

8. The method claim 1, wherein the air gap is 3 mm.

9. The method of claim 1, wherein the semi-stable fibers comprise an average diameter of from 0.1 μm to 10 μm.

10. The method of claim 1, wherein the hybrid fibrous scaffold comprises a thickness of from 0.01 mm to 1 mm.

11. The method of claim 1, wherein the hybrid fibrous scaffold comprises an average surface pore size of from 1 μm to 200 μm.

12. The method of claim 1, wherein the hybrid fibrous scaffold comprises a 90^{th} percentile scaffold pore size of greater than 25 μm.

13. The method of claim 1, wherein the hybrid fibrous scaffold comprises a structure that mimics an extracellular matrix of a subject.

14. The method of claim 1, wherein the polymer-containing solution is written in two or more layers, and wherein the predetermined writing path is different between the two or more layers.

15. The method of claim 1, wherein the solution comprises 1,1,1,3,3,3-hexafluoro-2-propanol (HFP).

16. The method of claim 1, wherein the polymer comprises polydioxanone, and the polymer is dissolved in the solution to a concentration of from 25 mg/mL to 450 mg/mL.

17. The method of claim 1, wherein
the step of electrically charging the polymer-containing solution comprises exposing the polymer-containing solution to an applied voltage;
the step of writing the polymer-containing solution comprises setting an air gap distance; and
the method further comprising increasing the number of the plurality of bent fibers by increasing the applied voltage, the air gap distance, or a combination thereof.

18. A hybrid fibrous scaffold, comprising:
a plurality of semi-stable fibers including a plurality of bent fibers and a plurality of straight fibers,
wherein the plurality of straight fibers are aligned to form a stacking grid geometry with a programmed grid spacing and the plurality of bent fibers extend across at least a portion of the programmed grid spacing.

19. The hybrid fibrous scaffold of claim 18, wherein the hybrid fibrous scaffold comprises a vascular graft hybrid fibrous scaffold.

20. The hybrid fibrous scaffold of claim 18, wherein the hybrid fibrous scaffold comprises a permeability to 9.9 μm microspheres of from 150 microspheres/mm² to 3000 microspheres/mm².

21. The hybrid fibrous scaffold of claim 18, wherein the hybrid fibrous scaffold comprises a permeability to 97 μm microspheres of from 1 microspheres/mm² to 5 microspheres/mm².

22. The hybrid fibrous scaffold of claim 18, wherein the scaffold comprises one or more therapeutic agents.

23. A method of promoting tissue regeneration or endothelialization in a subject, comprising:
providing a hybrid fibrous scaffold comprising semi-stable fibers including a plurality of bent fibers and a plurality of straight fibers; and
contacting the hybrid fibrous scaffold with tissue in the subject.
